SEQUENCE LISTING (5 sequences)

SEQ ID NO: 1 - protein, 104 aa, Rana pipiens
EDWLTFQKKHITNTRDVDCDNIMSTNLFHCKDKNTFIYSRPEPVKAICKGIIASKNVLTTSEFYLSDCNVTSRPCKYKLKKSTNKFCVTCENQAPVHFVGVGSC

SEQ ID NO: 2 - protein, 104 aa, Artificial Sequence
  ′805 variant of ranpirnase
EDWLTFQKKHVTNTRDVDCNNIMSTNLFHCKDKNTFIYSRPEPVKAICKGIIASKNVLTTSEFYLSDCNVTSRPCKYKLKKSTNKFCVTCENQAPVHFVGVGRC

SEQ ID NO: 3 - protein, 114 aa, Rana pipiens
KPKEDREWEKFKTKHITSQSVADFNCNRTMNDPAYTPDGQCKPVNTFIHSTTGPVKEICRRATGRVNKSSTQQFTLTTCKNPIRCKYSQSNTTNFICITCRDNYPVHFVKTGKC

SEQ ID NO: 4 - protein, 114 aa, Artificial Sequence
  rAmphirnase 2
KPKEDREWEKFKTKHITSQSVADFNCNRTMNDPAYTPDGQCKPINTFIHSTTGPVKEICRRATGRVNKSSTQQFTLTTCKNPIRCKYSQSNTTNFICITCRDNYPVHFVKTGKC

SEQ ID NO: 5 - protein, 104 aa, Rana pipiens
EDWLTFQKKHITNTRDVDCDNIMSSNLFHCKDKNTFIYSRPEPVKAICKGIIASKNVLTTSEFYLSDCNVTSRPCKYKLKKSTNKFCVTCENQAPVHFVGVGSC

(19) United States
(12) Patent Application Publication
(10) Pub. No.: US 2024/0123043 A1
(43) Pub. Date: Apr. 18, 2024

(54) PHARMACEUTICAL COMPOSITIONS COMPRISING RANPIRNASE

(71) Applicant: Orgenesis Inc., Germantown, MD (US)

(72) Inventor: Keith Arthur JOHNSON

(73) Assignee: Orgenesis Inc., Germantown, MD (US)

(21) Appl. No.: 18/547,164

(22) PCT Filed: Feb. 21, 2022
(86) PCT No.: PCT/US2022/017127

§ 371 Date: Aug. 21, 2023

Related U.S. Application Data
(60) Provisional application No. 63/151,802, filed on Feb. 22, 2021.

(51) Int. Cl. A61K 38/51; A61K 9/00; A61K 9/06; A61K 47/02; A61K 47/10; A61K 47/12; A61K 47/14; A61K 47/18; A61K 47/20; A61K 47/38; A61K 47/44

(52) U.S. Cl. CPC A61K 38/51; A61K 9/0014; A61K 9/06; A61K 47/02; A61K 47/10; A61K 47/12; A61K 47/14; A61K 47/183; A61K 47/20; A61K 47/38; A61K 47/44

(57) ABSTRACT

This disclosure is directed to new pharmaceutical compositions comprising ranpimase and a polyol, said compositions comprise increased stability. Further disclosed are methods for treating a viral disease by administering said compositions.

### FIELD OF THE DISCLOSURE

This disclosure is directed to new topical pharmaceutical compositions comprising ranpirnase and a polyol, said compositions comprise increased stability. Further disclosed are methods for treating a viral disease by administering said compositions.

### SEQUENCE LISTING STATEMENT

The instant application contains a Sequence Listing which has been submitted electronically in ASCII format and is hereby incorporated by reference in its entirety. Said ASCII copy, created on Feb. 18, 2021, is named P-597416-USP_ST25.txt and is 5284 bytes in size.

### BACKGROUND

Ribonucleases are a superfamily of enzymes that catalyze the degradation of RNA into smaller components. Ribonucleases are cytotoxic, as they cleave the RNA rendering indecipherable. Ranpirnase is a ribonuclease enzyme found in the oocytes of the Northern Leopard Frog (Rana pipiens). Ranpirnase degrades RNA substrates with a sequence preference for uracil and guanine nucleotides. Though not fully understood, at least part of ranpirnase mechanism of action is attributed to the RNA interference pathway, potentially through cleaving siRNA molecules, cleavage of transfer RNA, and interference with the NF-κB pathway.

Ranpirnase has demonstrated antitumor activity both in vitro and in vivo in several tumor models, being the only enzyme of this class that reached phase 3 clinical trials. Further, ranpirnase anti-viral activity makes it a candidate for treating several viral diseases, such as warts caused by different types of human papillomavirus (HPV).

Some dermal viruses might be preferably treated intralesionally. However, in situations as when lesions are small, too numerous, or located in areas where an injection would be painful, viral lesions should be treated topically.

Disclosed herein are pharmaceutical compositions comprising ranpirnase for topical delivery, said compositions showing increased stability, as well as other improved pharmacological properties, compared to existing ranpirnase formulations, comprising glycerol in a concentration above 10% of the weight of the formulation, such as K-Y Jelly®.

### SUMMARY OF THE INVENTION

In some aspects, disclosed herein is a pharmaceutical composition comprising ranpirnase and a polyol.

In some related aspects, the polyol is selected from a group comprising hexylene glycol, propylene glycol, glycerol, glycerol monolaureate, ethylene glycol, or any combination thereof. In some related aspects, the polyol comprises about 10% of the weight of the pharmaceutical composition.

In some related aspects, the glycerol comprises about 5%, and the propylene glycol comprises about 5% of the weight of the pharmaceutical composition. In some related aspects, the glycerol comprises about 5%, and the hexylene glycol comprises about 5% of the weight of the pharmaceutical composition.

In some related aspects, the pharmaceutical composition is stable at room temperature. In some related aspects, the pharmaceutical composition has increased stability compared to a composition comprising ranpirnase and about 10% glycerol of the weight of the pharmaceutical composition. In some related aspects, the composition is formulated as a gel or as a cream. In some related aspects, the composition is formulated for topical or dermal application. In some related aspects, the composition further comprises an antimicrobial agent. In some related aspects, the antimicrobial agent is selected from the group comprising methylparaben, propylparaben, EDTA, or any combination thereof.

In some related aspects, the composition does not comprise an antioxidant agent. In some related aspects, the composition comprises a pH in the range of 6.5 to 7. In some related aspects, the composition further comprises a surfactant agent. In some related aspects, the surfactant comprises sucrose stearate (SS), sodium dodecyl sulfate (SDS), sodium lauroyl sarcosinate (SLS), a combination of SS and SDS, or any combination thereof.

In some related aspects, the composition further comprises hydroxyethyl cellulose (HEC). In some related aspects, the composition further comprises cetostearyl alcohol, light mineral oil, octyldodecanol, or any combination thereof.

In some aspects, disclosed herein is a gel pharmaceutical composition comprising ranpirnase, glycerin, propylene glycol, hydroxyethyl cellulose, sodium citrate dihydrate, 10% hydrochloric acid, methylparaben, edetic acid, propylparaben and purified water.

In some aspects, disclosed herein is a gel pharmaceutical composition comprising ranpirnase, hydroxyethylcellulose, EDTA, methylparaben, propylparaben, glycerol, and propylene glycol, and/or hexylene glycol.

In some aspects, disclosed herein is a cream pharmaceutical composition comprising ranpirnase, sucrose stearate, sodium dodecyl sulfate, cetostearyl alcohol, EDTA, and light mineral oil, and/or octyldodecanol.

In some aspects, disclosed herein is a method for treating a viral disease in a subject in need thereof, the method comprising administering any of the pharmaceutical compositions, the gel pharmaceutical compositions, or the cream pharmaceutical compositions detailed above. In some relates aspects, the viral disease is selected from the group comprising a viral skin disease; herpes zoster; chickenpox; molluscum contagiosum; warts; measles; hand, foot and mouth disease, human papillomavirus (HPV) infection, or any combination thereof.

### BRIEF DESCRIPTION OF THE DRAWINGS

The subject matter disclosed herein is particularly pointed out and distinctly claimed in the concluding portion of the specification. The disclosure herein may best be understood by reference to the following detailed description when read with the accompanying drawings in which:

FIGS. 1A-1D show sample results of cation exchange chromatography (CEC) (FIG. 1A) and size exclusion chromatography (SEC) (FIG. 1B) of a standard composition comprising ranpirnase; as well as sample results of CEC (FIG. 1C) and SEC (FIG. 1D) of a standard composition comprising ranpirnase degraded by peroxides.

### DETAILED DESCRIPTION

In some embodiments, disclosed herein is a composition comprising a ribonuclease and a polyol. In some embodiments, disclosed herein is a composition comprising ranpirnase and a polyol.

### Ribonucleases

A skilled artisan would appreciate that ribonucleases, or RNases, are a type of nuclease that catalyzes the degradation of RNA into smaller components. RNases comprise a first defense against RNA viruses and provide the underlying machinery for more advanced cellular immune strategies such as RNAi. Several types of RNases can be used to degrade RNA, all of them can be used for the compositions and methods disclosed herein.

In some embodiments, the ribonuclease comprises ranpirnase. In some embodiments, the ribonuclease comprises onconase. In some embodiments, the ribonuclease comprises rAmphinase. In some embodiments, the ribonuclease comprises rAmphinase 2. In some embodiments, the ribonuclease comprises bovine seminal RNase (BS_RNase).

In some embodiments, the ribonuclease comprises a frog ribonuclease. In some embodiments, the ribonuclease comprises a frog oocytes ribonuclease. In some embodiments, the ribonuclease comprises an artificial ribonuclease. In some embodiments, more than one type of ribonuclease is used in the compositions and methods disclosed herein.

In some embodiments, the ribonuclease degrades tRNA. In some embodiments, the ribonuclease degrades rRNA. In some embodiments, the ribonuclease degrades mRNA. In some embodiments, the ribonuclease is conjugated to a molecule. In some embodiments, the ribonuclease is conjugated to human serum albumin.

In some embodiments, the ribonuclease comprises ranpirnase. A skilled artisan would appreciate that ranpirnase, called herein also “onconase”, “P-30”, “TMR004”, and “Pannon”, is a ribonuclease enzyme found in the oocytes of the Northern Leopard Frog (Rana pipiens). Ranpirnase is a member of the pancreatic ribonuclease (RNase A) protein superfamily and degrades RNA substrates with a sequence preference for uracil and guanine nucleotides. Ranpirnase has been studied as a potential cancer and antiviral treatment due to its unusual mechanism of cytotoxicity tested against transformed cells and antiviral activity. Ranpirnase UniProt identification number is P85073.

In some embodiments, ranpirnase comprises an amino acid sequence comprising EDWLTFQKKHITNTRDVDCDNIMSTNLFHCKDKNTFIYSRPEPVKAICKGIIASKN VLTTSEFYLSDCNVTSRPCKYKLKKSTNKFCVTCENQAPVHFVGVGSC (SEQ ID No.: 1). In some embodiments, ranpirnase comprises an amino acid sequence comprising at least 80%, 85%, 90%, 95%, or 99% homology to SEQ ID No.: 1.

In some embodiments, ranpirnase comprises an amino acid sequence comprising EDWLTFQKKHVTNTRDVDCNNIMSTNLFHCKDKNTFIYSRPEPVKAICKGIIASK NVLTTSEFYLSDCNVTSRPCKYKLKKSTNKFCVTCENQAPVHFVGVGRC (SEQ ID No.: 2). In some embodiments, ranpirnase comprises an amino acid sequence comprising at least 80%, 85%, 90%, 95%, or 99% homology to SEQ ID No.: 2.

In some embodiments, ranpirnase comprises a ranpirnase T25S isoform. In some embodiments, ranpirnase comprises an amino acid sequence comprising EDWLTFQKKHITNTRDVDCDNIMSSNLFHCKDKNTFIYSRPEPVKAICKGIIASKN VLTTSEFYLSDCNVTSRPCKYKLKKSTNKFCVTCENQAPVHFVGVGSC (SEQ ID No.: 5). In some embodiments, ranpirnase comprises an amino acid sequence comprising at least 80%, 85%, 90%, 95%, or 99% homology to SEQ ID No.: 5.

In some embodiments, the matured protein ranpirnase N-terminus glutamate is converted into pyroglutamic acid in the mature ranpirnase. In some embodiments, the compositions disclosed herein comprise ranpirnase comprising SEQ ID No.: 1, SEQ ID No.: 2, or SEQ ID No.: 5, but comprising pyroglutamic acid instead of glutamate in the N-terminus.

In some embodiments, the ribonuclease comprises amphinase. In some embodiments, disclosed herein is a composition comprising amphinase and immunoglobulins, fragments thereof, antibodies, or combinations thereof, obtained from a plasma of a subject immune to a viral disease. In some embodiments, disclosed herein is a composition comprising amphinase and immune cells. In some embodiments, disclosed herein is a composition comprising amphinase, immunoglobulins, fragments thereof, antibodies, or combinations thereof, obtained from a plasma of a subject immune to said viral disease, and immune cells. In some embodiments, disclosed herein is a composition comprising amphinase, immunoglobulins, fragments thereof, antibodies, or combinations thereof, obtained from a plasma of a subject immune to said viral disease, and natural killer cells.

A skilled artisan would appreciate that “amphinase”, termed herein also “amphinase 2” and “ramphinase”, is a ribonuclease enzyme found in the oocytes of the Northern leopard frog (Rana pipiens). Amphinase is a member of the pancreatic ribonuclease protein superfamily and degrades long RNA substrates, and has been studied as a potential cancer therapy due to its unusual mechanism of cytotoxicity tested against tumor cells.

In some embodiments, amphinase comprises an amino acid sequence comprising KPKEDREWEKFKTKHITSQSVADFNCNRTMNDPAYTPDGQCKPVNTFIHSTTGP VKEICRRATGRVNKSSTQQFTLTTCKNPIRCKYSQSNTTNFICITCRDNYPVHFVK TGKC (SEQ ID No.: 3). In some embodiments, amphinase comprises an amino acid sequence comprising at least 80%, 85%, 90%, 95%, or 99% homology to SEQ ID No.: 3.

In some embodiments, amphinase comprises an amino acid sequence comprising KPKEDREWEKFKTKHITSQSVADFNCNRTMNDPAYTPDGQCKPINTFIHSTTGPV KEICRRATGRVNKSSTQQFTLTTCKNPIRCKYSQSNTTNFICITCRDNYPVHFVKT GKC (SEQ ID No.: 4). In some embodiments, amphinase comprises an amino acid sequence comprising at least 80%, 85%, 90%, 95%, or 99% homology to SEQ ID No.: 4.

A skilled artisan would appreciate that ranpirnase and amphinase are RNAse A enzymes based on their amino acid sequence.

A skilled artisan would appreciate that different ribonucleases exert their antiviral activity by different mechanisms. All are relevant to the compositions and methods of the present disclosure. In some embodiments, a ribonuclease enters the cells via receptor-mediated endocytosis and once internalized into the cytosol, selectively degrades tRNA, resulting in inhibition of protein synthesis and induction of cell apoptosis.

A skilled artisan would appreciate that ribonucleases are hydrophilic compounds with relatively high stability. In some embodiments, the composition is encapsulated in naturally occurring lipid membranes.

### Viral Diseases

In some embodiments, disclosed herein are methods for treating a viral disease comprising providing a pharmaceutical composition comprising ranpirnase and a polyol. In some embodiments, disclosed herein are compositions comprising ranpirnase and a polyol for use in preparing a medicament for treating a viral disease. In some embodiments, the viral disease is caused by an adenovirus. In some embodiments, the viral disease is caused by a herpesvirus. In some embodiments, the viral disease is caused by a papillomavirus. In some embodiments, the viral disease is caused by a polyomavirus. In some embodiments, the viral disease is caused by a poxvirus. In some embodiments, the viral disease is caused by a hepadnavirus. In some embodiments, the viral disease is caused by a parvovirus. In some embodiments, the viral disease is caused by an astrovirus. In some embodiments, the viral disease is caused by SARS-COV-2.

In some embodiments, the viral disease is caused by a calicivirus. In some embodiments, the viral disease is caused by a picornavirus. In some embodiments, the viral disease is caused by a coronavirus. In some embodiments, the viral disease is caused by a flavivirus. In some embodiments, the viral disease is caused by a togavirus. In some embodiments, the viral disease is caused by a herpes virus. In some embodiments, the viral disease is caused by a retrovirus. In some embodiments, the viral disease is caused by an orthomyxovirus. In some embodiments, the viral disease is caused by an arenavirus. In some embodiments, the viral disease is caused by a bunyavirus.

In some embodiments, the viral disease is caused by a filovirus. In some embodiments, the viral disease is caused by a paramyxovirus. In some embodiments, the viral disease is caused by a rhabdovirus. In some embodiments, the viral disease is caused by a reovirus. In some embodiments, the viral disease is caused by Herpes simplex type 1. In some embodiments, the viral disease is caused by Herpes simplex type 2. In some embodiments, the viral disease is caused by Varicella-zoster virus.

In some embodiments, the viral disease is caused by Epstein-Barr virus. In some embodiments, the viral disease is caused by Human cytomegalovirus. In some embodiments, the viral disease is caused by human herpesvirus type 8. In some embodiments, the viral disease is caused by human papillomavirus. In some embodiments, the viral disease is caused by BK virus. In some embodiments, the viral disease is caused by JC virus. In some embodiments, the viral disease is caused by smallpox. In some embodiments, the viral disease is caused by Hepatitis B virus.

In some embodiments, the viral disease is caused by parvovirus B19. In some embodiments, the viral disease is caused by human astrovirus. In some embodiments, the viral disease is caused by Norwalk virus. In some embodiments, the viral disease is caused by coxsackievirus. In some embodiments, the viral disease is caused by hepatitis A virus. In some embodiments, the viral disease is caused by poliovirus. In some embodiments, the viral disease is caused by rhinovirus. In some embodiments, the viral disease is caused by severe acute respiratory syndrome virus. In some embodiments, the viral disease is caused by hepatitis C virus. In some embodiments, the viral disease is caused by yellow fever virus.

In some embodiments, the viral disease is caused by dengue virus. In some embodiments, the viral disease is caused by West Nile virus. In some embodiments, the viral disease is caused by TBE virus. In some embodiments, the viral disease is caused by Rubella virus. In some embodiments, the viral disease is caused by Hepatitis E virus. In some embodiments, the viral disease is caused by Human immunodeficiency virus (HIV). In some embodiments, the viral disease is caused by Influenza virus. In some embodiments, the viral disease is caused by Lassa virus. In some embodiments, the viral disease is caused by Crimean-Congo hemorrhagic fever virus.

In some embodiments, the viral disease is caused by Hantaan virus. In some embodiments, the viral disease is caused by Ebola virus. In some embodiments, the viral disease is caused by Marburg virus. In some embodiments, the viral disease is caused by Measles virus. In some embodiments, the viral disease is caused by Mumps virus. In some embodiments, the viral disease is caused by Parainfluenza virus. In some embodiments, the viral disease is caused by Respiratory syncytial virus. In some embodiments, the viral disease is caused by Rabies virus.

In some embodiments, the viral disease is caused by Hepatitis D. In some embodiments, the viral disease is caused by Rotavirus. In some embodiments, the viral disease is caused by Orbivirus. In some embodiments, the viral disease is caused by Coltivirus. In some embodiments, the viral disease is caused by Banna virus. In some embodiments, the viral disease is caused by more than one virus.

In some embodiments the viral disease is selected from the group comprising a viral skin disease; herpes zoster; chickenpox; molluscum contagiosum; warts; measles; hand, foot and mouth disease, human papillomavirus (HPV) infection, or any combination thereof.

In some embodiments, the viral disease comprises Covid-19. In some embodiments, the viral disease comprises acute hepatitis. In some embodiments, the viral disease comprises AIDS. In some embodiments, the viral disease comprises aseptic meningitis. In some embodiments, the viral disease comprises bronchiolitis. In some embodiments, the viral disease comprises Burkitt's lymphoma. In some embodiments, the viral disease comprises chickenpox. In some embodiments, the viral disease comprises chronic hepatitis.

In some embodiments, the viral disease comprises common cold. In some embodiments, the viral disease comprises congenital rubella. In some embodiments, the viral disease comprises congenital varicella syndrome. In some embodiments, the viral disease comprises congenital seizures in the newborn. In some embodiments, the viral disease comprises croup. In some embodiments, the viral disease comprises cystitis. In some embodiments, the viral disease comprises cytomegalic inclusion disease. In some embodiments, the viral disease comprises fatal encephalitis. In some embodiments, the viral disease comprises gastroenteritis.

In some embodiments, the viral disease comprises German measles. In some embodiments, the viral disease comprises gingivostomatitis. In some embodiments, the viral disease comprises hepatic cirrhosis. In some embodiments, the viral disease comprises hepatocellular carcinoma. In some embodiments, the viral disease comprises herpes labialis. In some embodiments, the viral disease comprises cold sores.

In some embodiments, the viral disease comprises herpes zoster. In some embodiments, the viral disease comprises Hodgkin's lymphoma. In some embodiments, the viral disease comprises hyperplastic epithelial lesions. In some embodiments, the viral disease comprises warts. In some embodiments, the viral disease comprises laryngeal papillomas.

In some embodiments, the viral disease comprises epidermodysplasia verruciformis. In some embodiments, the viral disease comprises infectious mononucleosis. In some embodiments, the viral disease comprises influenza. In some embodiments, the viral disease comprises influenza-like syndrome. In some embodiments, the viral disease comprises Kaposi sarcoma. In some embodiments, the viral disease comprises keratoconjunctivitis.

In some embodiments, the viral disease comprises liver disease. In some embodiments, the viral disease comprises lung and spleen diseases in the newborn. In some embodiments, the viral disease comprises malignancies. In some embodiments, the viral disease comprises cervical carcinoma. In some embodiments, the viral disease comprises squamous cell carcinomas. In some embodiments, the viral disease comprises measles. In some embodiments, the viral disease comprises multicentric Castleman disease.

In some embodiments, the viral disease comprises mumps. In some embodiments, the viral disease comprises myocarditis. In some embodiments, the viral disease comprises nasopharyngeal carcinoma. In some embodiments, the viral disease comprises pericarditis. In some embodiments, the viral disease comprises pharyngitis. In some embodiments, the viral disease comprises pharyngoconjunctival fever. In some embodiments, the viral disease comprises pleurodynia.

In some embodiments, the viral disease comprises pneumonia. In some embodiments, the viral disease comprises poliomyelitis. In some embodiments, the viral disease comprises postinfectious encephalomyelitis. In some embodiments, the viral disease comprises premature delivery. In some embodiments, the viral disease comprises primary effusion lymphoma. In some embodiments, the viral disease comprises rabies. In some embodiments, the viral disease comprises Reye syndrome.

In some embodiments, the viral disease comprises severe bronchiolitis with pneumonia. In some embodiments, the viral disease comprises skin vesicles. In some embodiments, the viral disease comprises mucosal ulcers. In some embodiments, the viral disease comprises tonsillitis. In some embodiments, the viral disease comprises pharyngitis. In some embodiments, the viral disease comprises Covid-19. In some embodiments, the viral disease is caused by SARS-COV-2. A skilled artisan will recognize that Covid-19, also termed “novel coronavirus pneumonia”, “NCP”, “SARS-COV-2 acute respiratory disease”, and “COVID-19” comprises an infectious respiratory disease caused by the 2019 novel coronavirus (SARS-COV-2), which was first detected during the 2019-20 Wuhan coronavirus outbreak. In some embodiments, SARS-COV-2 is transmitted through human-to-human transmission, generally via respiratory droplets as sneeze, cough or exhalation. In some embodiments, NCP symptoms appear after an incubation period of between 2 to 14 days. In some embodiments, coronavirus primarily affects the lower respiratory tract. In some embodiments, coronavirus primarily affects the upper respiratory tract. In some embodiments, NCP symptoms comprise fever, coughing, shortness of breath, pain in the muscles, tiredness, pneumonia, acute respiratory distress syndrome, sepsis, septic shock, death, or any combination thereof.

A skilled artisan will recognize that SARS-COV-2 belongs to the broad family of viruses known as coronaviruses. SARS-COV-2 is a positive-sense single-stranded RNA (+ssRNA) virus. SARS-COV-2 is a member of the subgenus Sarbecovirus (Beta-CoV lineage B), having an RNA sequence of approximately 30,000 bases in length. Eighty-one genomes of SARS-COV-2 had been isolated and reported. The present disclosure comprises compositions and methods for treating these SARS-COV-2 variants, or any further one.

A skilled artisan will recognize that seven coronavirus types are known to affect humans. The compositions and methods disclosed herein are useful for treating any of them. In some embodiments, the coronavirus comprises Human coronavirus 229E (HCoV-229E). In some embodiments, the coronavirus comprises Human coronavirus OC43 (HCoV-OC43). In some embodiments, the coronavirus comprises severe acute respiratory syndrome-related coronavirus (SARS-COV). In some embodiments, the coronavirus comprises Human coronavirus NL63 (HCoV-NL63, New Haven coronavirus). In some embodiments, the coronavirus comprises Human coronavirus HKUI. In some embodiments, the coronavirus comprises Middle East respiratory syndrome-related coronavirus (MERS-COV), previously known as novel coronavirus 2012 and HCoV-EMC. In some embodiments, the coronavirus comprises Novel coronavirus (SARS-COV-2), also known as Wuhan coronavirus.

### Methods of Treatment

In one embodiment, the compositions of the invention are used in the methods of the invention described herein. In one embodiment, the invention provides a method of preventing or treating a viral disease in a subject, comprising any of the compositions disclosed herein.

In some embodiments, the compositions and/or the formulations disclosed herein are used for treating a viral skin disease. In some embodiments, the compositions and/or the formulations disclosed herein are used for treating herpes zoster. In some embodiments, the compositions and/or the formulations disclosed herein are used for treating chickenpox. In some embodiments, the compositions and/or the formulations disclosed herein are used for treating molluscum contagiosum. In some embodiments, the compositions and/or the formulations disclosed herein are used for treating warts. In some embodiments, the compositions and/or the formulations disclosed herein are used for treating measles. In some embodiments, the compositions and/or the formulations disclosed herein are used for treating hand, foot and mouth disease.

In some embodiments, the compositions and formulations disclosed herein are used for treating a human papillomavirus (HPV) infection. A skilled artisan would appreciate that HPV infection is an infection caused by HPV, which is a DNA virus from the Papillomaviridae family. Though most HPV infections resolve spontaneously, a part of them persists, increasing the risk of cancer of the cervix, vulva, vagina, penis, anus, mouth, or throat. Over 170 types of HPV have been described. In some embodiments, the compositions disclosed herein are used for treating any of them.

In one embodiment, the term “treatment” refers to any process, action, application, therapy, or the like, wherein a subject, including a human being, is subjected to medical aid with the object of improving the subject's condition, directly or indirectly. In another embodiment, the term “treating” refers to reducing incidence, or alleviating symptoms, eliminating recurrence, preventing recurrence, preventing incidence, improving symptoms, improving prognosis or combinations thereof in other embodiments.

“Treating” embraces in another embodiment, the amelioration of an existing condition. The skilled artisan would understand that treatment does not necessarily result in the complete absence or removal of symptoms. Treatment also embraces palliative effects: that is, those that reduce the likelihood of a subsequent medical condition. The alleviation of a condition that results in a more serious condition is encompassed by this term.

As used herein, “subject” refers in one embodiment, to a human or any other animal which has been exposed to and is now immune to CoV related disease or Covid-2019. A subject refers to a human presenting to a medical provider for diagnosis or treatment of a disease, such as a CoV related disease or Covid-2019 in another embodiment. A human includes pre- and postnatal forms. In one embodiment, subjects are humans being treated for symptoms associated with a CoV related disease or Covid-2019.

The term “therapeutically effective amount” or “effective amount” refers in one embodiment, to an amount of a monovalent or combination vaccine sufficient to elicit a protective immune response in the subject to which it is administered. The immune response may comprise, without limitation, induction of cellular and/or humoral immunity.

The dosage regimen for treating a condition with the compositions of this invention is selected in one embodiment, in accordance with a variety of factors, such as the type, age, weight, ethnicity, sex and medical condition of the subject, the severity of the condition treated, and the particular compound employed, and thus may vary widely while still be in the scope of the invention.

### Pharmaceutical Compositions

In some embodiments, the compositions disclosed herein are delivered to the affected area of the skin in a pharmaceutically acceptable topical carrier. As used herein, a pharmaceutically acceptable topical carrier is any pharmaceutically acceptable formulation that can be applied to the skin surface for topical, dermal, intradermal, or transdermal delivery of a pharmaceutical or medicament.

In some embodiments, the compositions disclosed herein are formulated for topical application. In some embodiments, the compositions disclosed herein are formulated for dermal application. In some embodiments, the compositions disclosed herein are formulated for intradermal application. In some embodiments, the compositions disclosed herein are formulated for transdermal application.

In some embodiments, the pharmaceutical compositions disclosed herein comprise any pharmaceutically acceptable excipients. Acceptable excipients are well known in the art and listed for example in REMINGTON: THE SCIENCE AND PRACTICE OF PHARMACY 866-885(Alfonso R. Gennaro ed. 19th ed. 1995; Ghosh, T. K.; et al. TRANSDERMAL AND TOPICAL DRUG DELIVERY SYSTEMS (1997), which is incorporated hereby by reference, including, but not limited to, protectives, adsorbents, demulcents, antimicrobials, stabilizers, anti-oxidants, emollients, preservatives, antioxidants, moisturizers, buffering agents, solubilizing agents, skin-penetration agents, and surfactants.

In some embodiments, disclosed herein is a pharmaceutical composition comprising ranpirnase and a polyol. A skilled artisan would appreciate that a polyol comprises an organic compound containing multiple hydroxyl groups. In some embodiment, the polyol does not comprise functional groups other than hydroxyl groups. In some embodiments, the polyol comprises a water-soluble solvent. In some embodiments, the polyol comprises a polyol solvent. In some embodiments, the polyol comprises a semipolar solvent.

In some embodiments, the polyol comprises about 5% of the weight of the pharmaceutical composition. In some embodiments, the polyol comprises about 6% of the weight of the pharmaceutical composition. In some embodiments, the polyol comprises about 7% of the weight of the pharmaceutical composition. In some embodiments, the polyol comprises about 8% of the weight of the pharmaceutical composition. In some embodiments, the polyol comprises about 9% of the weight of the pharmaceutical composition. In some embodiments, the polyol comprises about 10% of the weight of the pharmaceutical composition. In some embodiments, the polyol comprises from about 11% to about 20% of the weight of the pharmaceutical composition.

In some embodiments, the polyol is selected from a group comprising hexylene glycol, propylene glycol, glycerol, glycerol monolaureate, and ethylene glycol, or any combination thereof. In some embodiments, the polyol comprises hexylene glycol. In some embodiments, the polyol comprises propylene glycol. In some embodiments, the polyol comprises glycerol. In some embodiments, the polyol comprises glycerol monolaureate. In some embodiments, the polyol comprises ethylene glycol.

In some embodiments, the glycerol comprises from about 0% to about 5% of the weight of the pharmaceutical composition. In some embodiments, the glycerol comprises about 5% of the weight of the pharmaceutical composition. In some embodiments, the glycerol comprises about 6% of the weight of the pharmaceutical composition. In some embodiments, the glycerol comprises about 7% of the weight of the pharmaceutical composition. In some embodiments, the glycerol comprises about 8% of the weight of the pharmaceutical composition. In some embodiments, the glycerol comprises about 9% of the weight of the pharmaceutical composition. In some embodiments, the glycerol comprises about 10% of the weight of the pharmaceutical composition.

In some embodiments, the glycerol comprises about 11% of the weight of the pharmaceutical composition. In some embodiments, the glycerol comprises about 12% of the weight of the pharmaceutical composition. In some embodiments, the glycerol comprises about 13% of the weight of the pharmaceutical composition. In some embodiments, the glycerol comprises about 14% of the weight of the pharmaceutical composition. In some embodiments, the glycerol comprises about 15% of the weight of the pharmaceutical composition. In some embodiments, the glycerol comprises from about 16% to about 20% of the weight of the pharmaceutical composition. In some embodiments, the glycerol comprises more than 20% of the weight of the pharmaceutical composition.

In some embodiments, the propylene glycol comprises about 0% of the weight of the pharmaceutical composition. In some embodiments, the propylene glycol comprises about 1% of the weight of the pharmaceutical composition. In some embodiments, the propylene glycol comprises about 2% of the weight of the pharmaceutical composition. In some embodiments, the propylene glycol comprises about 3% of the weight of the pharmaceutical composition. In some embodiments, the propylene glycol comprises about 4% of the weight of the pharmaceutical composition. In some embodiments, the propylene glycol comprises about 5% of the weight of the pharmaceutical composition. In some embodiments, the propylene glycol comprises about 6% of the weight of the pharmaceutical composition. In some embodiments, the propylene glycol comprises about 7% of the weight of the pharmaceutical composition. In some embodiments, the propylene glycol comprises about 8% of the weight of the pharmaceutical composition. In some embodiments, the propylene glycol comprises about 9% of the weight of the pharmaceutical composition. In some embodiments, the propylene glycol comprises about 10% of the weight of the pharmaceutical composition.

In some embodiments, the propylene glycol comprises from about 11% to about 20% of the weight of the pharmaceutical composition. In some embodiments, the propylene glycol comprises more than 20% of the weight of the pharmaceutical composition.

In some embodiments, the hexylene glycol comprises about 0% of the weight of the pharmaceutical composition. In some embodiments, the hexylene glycol comprises about 1% of the weight of the pharmaceutical composition. In some embodiments, the hexylene glycol comprises about 2% of the weight of the pharmaceutical composition. In some embodiments, the hexylene glycol comprises about 3% of the weight of the pharmaceutical composition. In some embodiments, the hexylene glycol comprises about 4% of the weight of the pharmaceutical composition. In some embodiments, the hexylene glycol comprises about 5% of the weight of the pharmaceutical composition. In some embodiments, the hexylene glycol comprises about 6% of the weight of the pharmaceutical composition. In some embodiments, the hexylene glycol comprises about 7% of the weight of the pharmaceutical composition. In some embodiments, the hexylene glycol comprises about 8% of the weight of the pharmaceutical composition. In some embodiments, the hexylene glycol comprises about 9% of the weight of the pharmaceutical composition. In some embodiments, the hexylene glycol comprises about 10% of the weight of the pharmaceutical composition.

In some embodiments, the hexylene glycol comprises from about 11% to about 20% of the weight of the pharmaceutical composition. In some embodiments, the hexylene glycol comprises more than 20% of the weight of the pharmaceutical composition.

In some embodiments, the glycerol comprises about 5%, and the propylene glycol comprises about 5% of the weight of the pharmaceutical composition. In some embodiments, the glycerol comprises about 5%, and the hexylene glycol comprises about 5% of the weight of the pharmaceutical composition.

In some embodiments, the polyol comprises a polyglycol. As used herein, the term polyglycol refers to a compound of the ether-glycol type, which contains several ether linkages that yields one or more glycols on hydrolysis of these linkages.

In some embodiments, the polyol comprises a polyhexylene glycol. A skilled artisan would appreciate that polyhexylene glycol is a polymeron comprising hexylene glycols, also termed 2-Methyl-2,4-pentanediol (MPD), is an organic compound with the formula (CH₃)₂C(OH)CH₂CH(OH)CH₃. This colorless liquid is a chiral diol.

In some embodiments, the polyol comprises polypropylene glycol. A skilled artisan would appreciate that polypropylene glycol, also termed polypropylene oxide, is the polymer of propylene glycol, wherein propylene glycol has the formula CH₃CH(OH)CH₂OH. Polypropylene glycol is a polyether, and more generally it is a polyalkylene glycol (PAG).

In some embodiments, the polyol comprises polyglycerol. A skilled artisan would appreciate that polyglycerol, also termed polyglycerine, is a polymer comprising glycerol, which is a simple polyol compound, colorless, odorless, viscous liquid that is sweet-tasting and non-toxic. The glycerol backbone is found in those lipids known as glycerides. Owing to the presence of three hydroxyl groups, glycerol is miscible with water and is hygroscopic in nature. Glycerol has the formula C₃H₈O₃.

In some embodiments, the polyol comprises polyglycerol monolaurate. A skilled artisan would appreciate that polyglycerol monolaurate (GML), also known as monolaurin, glyceryl laurate or 1-lauroyl-glycerol, is a polymer comprising glycol monolaureate, which is a monoglyceride. It is the mono-ester formed from glycerol and lauric acid. Its chemical formula is C₁₅H₃₀O₄.

In some embodiments, the polyglycerol comprises polyglycerol-3-oleate. A skilled artisan would appreciate that polyglycerol-3-oleate is a mono-ester of oleic acid and a glycerin polymer containing an average of three glycerin units.

In some embodiments, the polyol comprises polyethylene glycol. A skilled artisan would appreciate that polyethylene glycol (PEG), also known as polyethylene oxide (PEO) or polyoxyethylene (POE), is used as an excipient in many pharmaceutical products. PEG has the formula C₂ₙH₄ₙ₊₂Oₙ₊₁.

In some embodiments, the pharmaceutical composition is stable at room temperature. A skilled artisan would appreciate that the term “room temperature” identifies as around 20-22° C. In some embodiments, the pharmaceutical composition has increased stability compared to a composition comprising ranpirnase and about 10% glycerol of the weight of said pharmaceutical composition. A skilled artesian would appreciate that stability is assets by no significant degradation, no significant trend down in assay values, no new impurities observed, area appearance or any combination thereof.In some embodiments, the pharmaceutical compositions disclosed herein comprise a preservative. A skilled artisan would appreciate that the term preservative refers to agents used to prevent decomposition by microbial growth or by other undesirable chemical changes. In some embodiments, the preservative comprises an antifungal. In some embodiments, the preservative comprises an antimicrobial.

In some embodiments, the antimicrobial agent is selected from the group comprising methylparaben, propylparaben, EDTA, or any combination thereof. In some embodiments, the antimicrobial comprises methylparaben. In some embodiments, the antimicrobial comprises propylparaben. In some embodiments, the antimicrobial comprises EDTA. In some embodiments, the preservative comprises benzyl alcohol. In some embodiments, the antimicrobial comprises antibacterial esters, esters of parahydroxybenzoic acid, chlorhexidine, chlorocresol, benzoic acid, and polymyxin, or any combination thereof. In some embodiments, the antimicrobial agent comprises a combination of antimicrobial compounds.

In some embodiments, the antimicrobial agent comprises a combination of methylparaben and propylparaben. In some embodiments, the antimicrobial agent comprises a combination of methylparaben, propylparaben, and EDTA.

In some embodiments, the preservative is selected from a group comprising quaternary ammonium compounds, such as benzalkonium chloride, benzethonium chloride, cetrimide, dequalinium chloride, and cetylpyridinium chloride; mercurial agents, such as phenylmercuric nitrate, phenylmercuric acetate, and thimerosal; alcoholic agents, for example, chlorobutanol, phenylethyl alcohol, and benzyl alcohol.

In some embodiments, the pharmaceutical compositions disclosed herein does not comprise an antioxidant agent. In some embodiments, the pharmaceutical compositions disclosed herein comprise an antioxidant agent. In some embodiments, the antioxidant agent is selected from a group comprising ascorbic acid and its esters, sodium bisulfite, butylated hydroxytoluene, butylated hydroxyanisole, tocopherols, chelating agents like EDTA, citric acid or any combination thereof.

In some embodiments, the pH of the pharmaceutical compositions disclosed herein is within the range of from about 6 to about 7.5. In some embodiments, the pH of the pharmaceutical compositions disclosed herein is within the range of from about 6.5 to about 7. In some embodiments, the pH is about 6.5, 6.6, 6.7, 6.8, 6.9, or 7. In some embodiments, an effective amount of a buffer agent is included to stabilize the pH. In one embodiment, the buffering agent is present in the aqueous topical formulation in an amount of from about 0.05 to about 1 weight percent of the formulation. Acids or bases can be used to adjust the pH as needed. In some embodiments, the buffering agent comprises phosphate buffer. In some embodiments, the buffering agent comprises citrate buffer. In some embodiments, the buffering agent comprises acetate buffer. In some embodiments, the buffering agent comprises a lactic acid buffer. In some embodiments, the buffering agent comprises a borate buffer.

In some embodiments, the buffer agent comprises sodium chloride, potassium chloride, mannitol, dextrose, glycerol, propylene glycol, or any combination thereof. A skilled artisan would appreciate that the concentration of the buffering agent can vary widely depending on the formulation's desired properties. In some embodiments, the buffer agent comprises from about 0.5 to about 1 weight percent of the formulation.

In some embodiments, the pharmaceutical compositions disclosed herein comprise a surfactant agent. A skilled artisan will appreciate that surfactants are compounds that lower the surface tension between two liquids, or between a liquid and a solid. Further, a in some embodiments, the surfactant mitigates the propensity of molecules, for example proteins to aggregate. Surfactants additives are particularly useful if a pump or plastic container is used to administer the pharmaceutical compositions.

In some embodiments, the surfactant comprises sucrose stearate (SS), sodium dodecyl sulfate (SDS), sodium lauroyl sarcosinate (SLS), a combination of SS and SDS, or any combination thereof.

In some embodiments, the surfactant comprises sucrose stearate (SS). A skilled artisan will appreciate that sucrose stearate is a mixture of sucrose and stearic acid. Sucrose stearate is used as an additive or supportive ingredient in skin care products, as the rich texture that allows sucrose stearate to function well as an emollient also makes it useful as a thickener in skin care products and cosmetics.

In some embodiments, the surfactant comprises sodium dodecyl sulfate (SDS). A skilled artisan will appreciate that SDS is a synthetic organic compound with the formula CH₃(CH₂)₁₁SO₄Na.

In some embodiments, the surfactant comprises sodium lauroyl sarcosinate (SLS). A skilled artisan will appreciate that SLS, also known as sarkosyl, is an anionic surfactant derived from sarcosine, used in diverse pharmaceutical formulations. SLS has the formula C₁₅H₂₈NNaO₃.

In some embodiments, the surfactant comprises a combination of SS and SDS.

In some embodiments, the pharmaceutical compositions of the present disclosure comprise a formulation for topically or dermally administered pharmaceuticals. A skilled artisan would appreciate that topical administration relates to applying to a particular place on or in the body. Most often topical administration means application to body surfaces such as the skin or mucous membranes. A skilled artisan would further appreciate that dermal application relates to application to the relevant skin layers.

In some embodiments, the pharmaceutical compositions are formulated as a gel or as a cream. In some embodiments, the pharmaceutical compositions are formulated as a gel. In some embodiments, the pharmaceutical compositions are formulated as a cream. In some embodiments, the pharmaceutical compositions comprise a gel. In some embodiments, the pharmaceutical compositions comprise a pharmaceutically acceptable solvent, such as a polyalcohol or water. In some embodiments, the pharmaceutical compositions comprise an emulsion, either an oil-in-water or a water-in-oil emulsions. In some embodiments, the pharmaceutical compositions comprise a lotion. In some embodiments, the pharmaceutical compositions comprise a micro emulsion. In some embodiments, the pharmaceutical compositions comprise an ointment. In some embodiments, the pharmaceutical compositions comprise liposomes. In some embodiments, the pharmaceutical compositions comprise a powder. In some embodiments, the pharmaceutical compositions comprise an aqueous solution. In some embodiments, the pharmaceutical compositions comprise a suspension.

In some embodiments, the pharmaceutical compositions disclosed herein comprises a gel, for example, a two-phase gel or a single-phase gel. A skilled artisan would appreciate that gels are semisolid systems consisting of suspensions of small inorganic particles or large organic molecules interpenetrated by a liquid. When the gel mass comprises a network of small discrete inorganic particles, it is classified as a two-phase gel. Single-phase gels consist of organic macromolecules distributed uniformly throughout a liquid such that no apparent boundaries exist between the dispersed macromolecules and the liquid. Suitable gels for use in the invention are disclosed in REMINGTON: THE SCIENCE AND PRACTICE OF PHARMACY 1517-1518 (Alfonso R. Gennaro ed. 19th ed. 1995), incorporated herein by reference.

A skilled artisan would appreciate that many gelling agents or structure forming ingredients known in the art can be applied to the compositions disclosed herein in order to obtain a gel formulation. In some embodiments, a gelling agent comprises a hydrophilic or a hydroalcoholic gelling agent. In some embodiments, the gelling agent comprises between about 0.2% to about 4% of the composition weight.

In some embodiments, the pharmaceutical composition comprises hydroxyethylcellulose (HEC). In some embodiments, the pharmaceutical composition comprises less than about 1% HEC. In some embodiments, the pharmaceutical composition comprises from about 1% to about 2% HEC. In some embodiments, the pharmaceutical composition comprises 1.75% HEC. In some embodiments, the pharmaceutical composition comprises more than 2% HEC.

In some embodiments, the pharmaceutical composition comprises a gelling agent selected from the group comprising carbomer, cellulose gum, MVE/MA decadiene crosspolymer, PVM/MA copolymer, hydroxyethylcellulose (HEC), or any combination thereof.

In some embodiments, the pharmaceutical compositions of the invention are formulated as an emulsion. A skilled artisan would appreciate that an emulsion refers to a dispersed system comprising at least two immiscible phases, one phase dispersed in the other as droplets ranging in diameter from 0.1 μm to 100 μm. In some embodiments, an emulsifying agent is included to improve stability. Emulsions, such as creams and lotions that can be used as topical carriers and their preparation are disclosed in REMINGTON: THE SCIENCE AND PRACTICE OF PHARMACY 282-291 (Alfonso R. Gennaro ed. 19th ed. 1995), hereby incorporated herein by reference.

In some embodiments, the pharmaceutical compositions disclosed herein comprises a cream. A skilled artisan would appreciate that the term cream refers to semi-solid emulsions of oil and water. In some embodiments, a cream is an oil-in-water (O/W) cream, composed of small droplets of oil dispersed in a continuous water phase. In some embodiments, a cream is a water-in-oil (W/O) cream, composed of small droplets of water dispersed in a continuous oily phase.

In some embodiment, the cream comprises a structure forming ingredient. In some embodiments the structure forming ingredient of a cream comprises sodium dodecyl sulfate (SDS). In some embodiments the structure forming ingredient of a cream comprises sucrose stearate (SS). In some embodiments, the structure forming ingredient of a cream comprises a combination of SS and SDS. In some embodiments, the SS comprises about 1.5% and the SDS comprises about 0.2% of the weight of the pharmaceutical composition. In some embodiments, the SS comprises less than 1% of the weight of the pharmaceutical composition. In some embodiments, the SS comprises between about 1% and about 2% of the weight of the pharmaceutical composition. In some embodiments, the SS comprises between about 1.5% of the weight of the pharmaceutical composition. In some embodiments, the SS comprises over 2% of the weight of the pharmaceutical composition.

In some embodiments, the SDS comprises less than 0.1% of the weight of the pharmaceutical composition. In some embodiments, the SDS comprises between about 0.1% and about 0.3% of the weight of the pharmaceutical composition. In some embodiments, the SS comprises between about 0.2% of the weight of the pharmaceutical composition. In some embodiments, the SS comprises over 0.2% of the weight of the pharmaceutical composition.

In some embodiments, the pharmaceutical composition comprises cetostearyl alcohol. In some embodiments, the composition comprises less than 5% cetostearyl alcohol. In some embodiments, the composition comprises between about 5% to about 10% cetostearyl alcohol. In some embodiments, the composition comprises 8% cetostearyl alcohol. In some embodiments, the composition comprises over 10% cetostearyl alcohol.

In some embodiments, the pharmaceutical composition comprises light mineral oil. In some embodiments, the composition comprises less than 2.5% light mineral oil. In some embodiments, the composition comprises between about 2.5% to about 7.5% light mineral oil. In some embodiments, the composition comprises 5% light mineral oil. In some embodiments, the composition comprises over 7.5% light mineral oil.

In some embodiments, the pharmaceutical composition comprises octyldodecanol. In some embodiments, the composition comprises less than 2.5% octyldodecanol. In some embodiments, the composition comprises between about 2.5% to about 15% octyldodecanol. In some embodiments, the composition comprises 5% octyldodecanol. In some embodiments, the composition comprises 12% octyldodecanol. In some embodiments, the composition comprises over 15% octyldodecanol.

In some embodiments, the composition further comprises cetostearyl alcohol, light mineral oil, octyldodecanol or any combination thereof.

In some embodiments, the composition comprises a combination of cetostearyl alcohol and light mineral oil. In some embodiments, the composition comprises a combination of cetostearyl alcohol and octyldodecanol.

In some embodiments, the cream pharmaceutical composition comprises semipolar solvents. In some embodiments, the semipolar solvents comprise propylene glycol, hexylene glycol or combination thereof.

In some embodiments, the pharmaceutical composition disclosed herein is formulated as an ointment. A skilled artisan would appreciate that an ointment refers to an oleaginous semisolid that contains little if any water. In some embodiments, the ointment is hydrocarbon based, such as a wax, petrolatum, or gelled mineral oil.

In some embodiments, disclosed herein is a pharmaceutical composition comprising ranpirnase, hydroxyethylcellulose, EDTA, methylparaben, propylparaben, glycerol, and propylene glycol. In some embodiments, disclosed herein is a pharmaceutical composition comprising ranpirnase, hydroxyethylcellulose, EDTA, methylparaben, propylparaben, glycerol, and hexylene glycol.

In some embodiments, disclosed herein is a pharmaceutical composition comprising ranpirnase, sucrose stearate, sodium dodecyl sulfate, cetostearyl alcohol, EDTA, and light mineral oil. In some embodiments, disclosed herein is a pharmaceutical composition comprising ranpirnase, sucrose stearate, sodium dodecyl sulfate, cetostearyl alcohol, EDTA, and octyldodecanol.

In some embodiments, disclosed herein is a gel pharmaceutical composition comprising ranpirnase, glycerin, propylene glycol, hydroxyethyl cellulose, sodium citrate dihydrate, 10% hydrochloric acid, methylparaben, edetic acid, propylparaben and purified water.

In some embodiments, disclosed herein is a gel pharmaceutical composition comprising 25.0% w/w ranpirnase bulk drug substance solution (BDSS), 5.0% w/w glycerin, 5.0% w/w propylene glycol, 1.75% w/w hydroxyethyl cellulose, 0.42% w/w sodium citrate dihydrate, 0.21% w/w 10% hydrochloric acid, 0.11% w/w methylparaben, 0.1% w/w edetic acid, 0.022% w/w propylparaben and Q.S. TO 100% purified water.

In one embodiment, ranpirnase bulk drug substance solution (BDSS) comprises 4 mg/mL ranpirnase.

In some embodiments, disclosed herein is a gel pharmaceutical composition comprising ranpirnase, hydroxyethylcellulose, EDTA, methylparaben, propylparaben, glycerol, and propylene glycol, and/or hexylene glycol.

In some embodiments, disclosed herein is a cream pharmaceutical composition comprising ranpirnase, sucrose stearate, sodium dodecyl sulfate, cetostearyl alcohol, EDTA, and light mineral oil, and/or octyldodecanol.

In some embodiments, the pharmaceutical compositions comprise protectives and adsorbents. In some embodiments, protectives and adsorbents are selected from a group comprising dusting powders, zinc sterate, collodion, dimethicone, silicones, zinc carbonate, aloe vera gel and other aloe products, vitamin E oil, allatoin, glycerol, petrolatum, zinc oxide, or any combination thereof.

In some embodiments, the pharmaceutical compositions comprise demulcents. In some embodiments, demulcents comprise benzoin, hydroxypropyl cellulose, hydroxypropyl methylcellulose, polyvinyl alcohol or any combination thereof. Suitable emollients include, but are not limited to, animal and vegetable fats and oils, myristyl alcohol, alum, and aluminum acetate.

In some embodiments, the pharmaceutical compositions disclosed herein comprise a moisturizer. In some embodiments, a moisturizer comprises glycerol, sorbitol, polyethylene glycols, urea, propylene glycol or any combinations thereof.

In some embodiments, the pharmaceutical compositions disclosed herein comprise a solubilizing agent. In some embodiments, a solubilizing agent comprises quaternary ammonium chlorides, cyclodextrins, benzyl benzoate, lecithin, and polysorbates.

In some embodiments, the pharmaceutical compositions disclosed herein comprise a skin-penetration agent. In some embodiments, the skin penetration agent comprises ethyl alcohol, isopropyl alcohol, octylphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate); and N-methyl pyrrolidone, or any combination thereof.

In some embodiments, the pharmaceutical compositions disclosed herein comprise active ingredients other than ranpirnase. In some embodiments, the active ingredient is selected from the group comprising topical corticosteroids, such as betamethasone, diflorasone, amcinonide, fluocinolone, mometasone, hydrocortisone, prednisone, and triamcinolone; local anesthetics and analgesics, such as camphor, menthol, lidocaine, and dibucaine, and pramoxine; antifungals, such as ciclopirox, chloroxylenol, triacetin, sulconazole, nystatin, undecylenic acid, tolnaftate, miconizole, clotrimazole, oxiconazole, griseofulvin, econazole, ketoconozole, and amphotericin B; antibiotics and anti-infectives, such as mupirocin, erythromycin, clindamycin, gentamicin, polymyxin, bacitracin, and silver sulfadiazine; and antiseptics, such as iodine, povidine-iodine, benzalkonium chloride, benzoic acid, chlorhexidine, nitrofurazine, benzoyl peroxide, hydrogen peroxide, hexachlorophene, phenol, resorcinol, and cetylpyridinium chloride; or any combination thereof.

In some embodiments, the pharmaceutical compositions disclosed herein comprise about 0.1% w/w ranpirnase. In some embodiments, the pharmaceutical compositions comprise about 0.01% w/w ranpirnase. In some embodiments, the pharmaceutical compositions comprise about 0.03% w/w ranpirnase. In some embodiments, the pharmaceutical compositions comprise about 0.05% w/w ranpirnase. In some embodiments, the pharmaceutical compositions comprise about 0.07% w/w ranpirnase. In some embodiments, the pharmaceutical compositions comprise about 0.09% w/w ranpirnase. In some embodiments, the pharmaceutical compositions comprise about 0.12% w/w ranpirnase. In some embodiments, the pharmaceutical compositions comprise about 0.14% w/w ranpirnase. In some embodiments, the pharmaceutical compositions comprise about 0.16% w/w ranpirnase. In some embodiments, the pharmaceutical compositions comprise about 0.18% w/w ranpirnase.

In some embodiments, the pharmaceutical compositions disclosed herein comprise about 0.001% w/w ranpirnase. In some embodiments, the pharmaceutical compositions disclosed herein comprise about 0.002% w/w ranpirnase. In some embodiments, the pharmaceutical compositions disclosed herein comprise about 0.003% w/w ranpirnase. In some embodiments, the pharmaceutical compositions disclosed herein comprise about 0.004% w/w ranpirnase. In some embodiments, the pharmaceutical compositions disclosed herein comprise about 0.005% w/w ranpirnase. In some embodiments, the pharmaceutical compositions disclosed herein comprise about 0.006% w/w ranpirnase. In some embodiments, the pharmaceutical compositions disclosed herein comprise about 0.007% w/w ranpirnase. In some embodiments, the pharmaceutical compositions disclosed herein comprise about 0.008% w/w ranpirnase. In some embodiments, the pharmaceutical compositions disclosed herein comprise about 0.009% w/w ranpirnase.

In some embodiments, the pharmaceutical compositions disclosed herein comprise about 0.2% w/w ranpirnase. In some embodiments, the pharmaceutical compositions disclosed herein comprise about 0.25% w/w ranpirnase. In some embodiments, the pharmaceutical compositions disclosed herein comprise about 0.31% w/w ranpirnase. In some embodiments, the pharmaceutical compositions disclosed herein comprise about 0.3% w/w ranpirnase. In some embodiments, the pharmaceutical compositions comprise about 0.4% w/w ranpirnase. In some embodiments, the pharmaceutical compositions comprise about 0.6% w/w ranpirnase. In some embodiments, the pharmaceutical compositions comprise about 0.8% w/w ranpirnase. In some embodiments, the pharmaceutical compositions comprise about 1% w/w ranpirnase.

In some embodiments, the pharmaceutical compositions disclosed herein are used in combination with treatment regimens and medications for treatment of dermatologic disorders known in the art, as for example those disclosed in THE MERCK MANUAL 811-830 (Keryn A. G. Lane et al. eds. 17th ed. 2001), which is incorporated herein by reference.

A skilled artisan will appreciate that combining the pharmaceutical compositions with other treatments and medications comprises administering both compounds to a subject within a time interval such that they can act together to treat and/or prevent a medical condition. A skilled artisan will appreciate that any suitable route of administration can be employed to deliver the additional treatment or medication, comprising oral, intraoral, rectal, parenteral, topical, epicutaneous, transdermal, subcutaneous, intramuscular, intranasal, sublingual, buccal, intradural, intraocular, intrarespiratory, nasal inhalation, or any combination thereof.

In some embodiments, the pharmaceutical compositions disclosed herein are used in combination with systemic administration of antibiotics or retinoids, such as tetracycline, minocin, minocycline, erythromycin, doxycycline, isotretinoins, or any combination thereof.

In some embodiments, the pharmaceutical compositions disclosed herein are used in combination with a topical treatment. In some embodiments, a topical treatment comprises metronidizole, hydrogen peroxide, benzoyl peroxide, lipoic acid, azelaic acid, sulfur preparations; topically dosed antibiotics, metronidazole, clindamycin, and erythromycin; topical retinoids, tretinoin, adapalene, tazarotene; or topical steroids.

A skilled artisan will appreciate that dosages and dosing frequency are determined by physician or a pharmacologist according to methods well known in the art. These methods should consider the activity of the compositions, the characteristics of the formulation, and the characteristics of the dermatologic disorder to be treated or prevented.

The term “about” as used herein means in quantitative terms plus or minus 5%, or in another embodiment plus or minus 10%, or in another embodiment plus or minus 15%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the broad scope of the invention.

### EXAMPLES

### Example 1

### Ranpirnase Formulation—Materials and Methods

Materials: Ranpirnase was provided either in a frozen solution at a ˜4 mg/mL concentration, or as a lyophilized solid at a ˜1 mg/vial concentration. All excipients used for preformulation and formulation work were from the United States Pharmacopeia and National Formulary (USP/NF), if a monograph existed. All solvents and chemicals for analytical work were HPLC or reagent grade.

Methods: HPLC analysis was performed with an Agilent 1200 instrument using a diode array detector. An Orion 710A+ pH meter with a Thermo Scientific electrode was used for pH measurement. For small solution volumes, test strips (Precision Laboratories, Inc.) were used to measure pH to within 0.5 units. Viscosity measurements were performed with a Brookfield rotational viscometer according to the details below:

Hydroxyethylcellulose gels: RV viscometer, Spindle #14, 12 rpm.

Creams: LV or RV viscometer, Helipath stand, Spindle #92, 1.5 rpm

For compounding, low shear mixing was performed with a stainless-steel propeller blade (1.5″ diameter) and an IKA Eurostar 200 overhead mixer. High shear mixing was performed with a GLH Homogenizer using a 10 mm stainless steel rotor-stator head. Further details on compounding are described later in the following examples.

The chromatography conditions used for ranpirnase analysis are summarized in Table 1.

TABLE 1
Chromatography conditions.

| | Cation Exchange | Size Exclusion |
|---|---|---|
| | Chromatography | Chromatography |
| | (CEC) | (SEC) |
| Column | Mono S 5/50 GL (GE) | OHpak SB-803 HQ |
| | | (Shodex) |
| Sample | Ambient | Ambient |
| Temperature | | |
| Column | Ambient | Ambient |
| Temperature | | |
| Injection | 25 μL for 1 mg/mL solution | 25 μL |
| Volume | 100 μL for 0.1 mg/mL extracts | |
| Flow Rate | 0.8 mL/min | 0.5 mL/min |
| Detection | 280 nm | 280 nm |
| Mobile Phase | A: 50 mM MES buffer (pH 6.75) | 100 mM phosphate |
| | B: 50 mM MES buffer (pH 6.75) | with 83 mM Na |
| | with 0.2M NaCl | sulfate (pH 7.2) |
| Gradient | 0 min: 10% B | Isocratic |
| | 35 min: 75% B | |
| | 37 min: 75% B | |
| | 37.1 min: 10% B | |
| Run time | 45 min | 35 min |

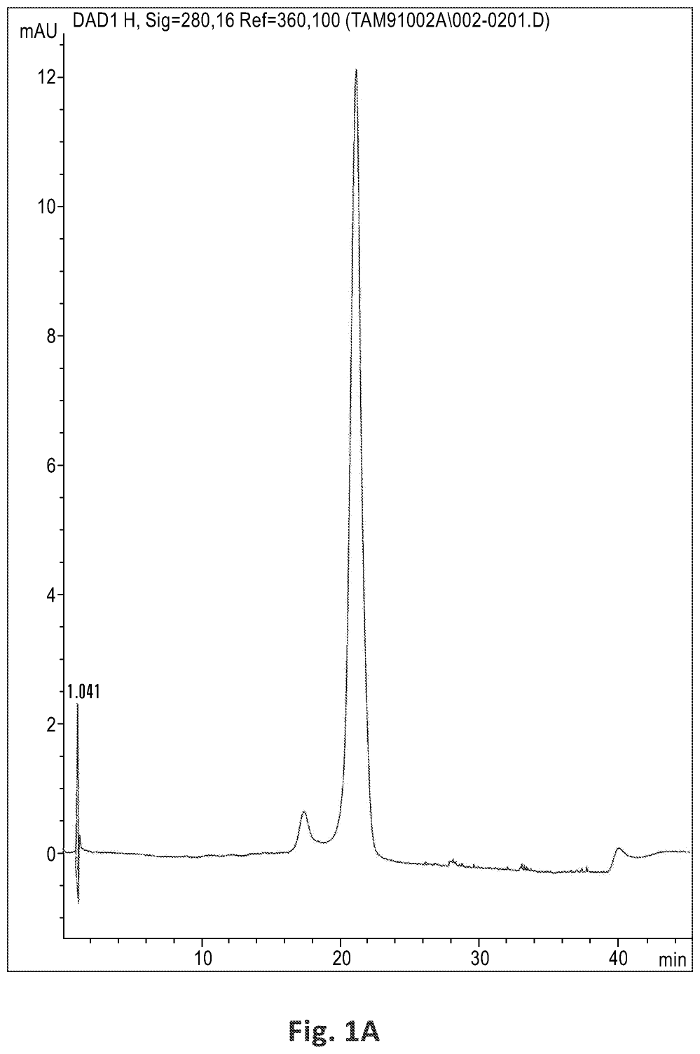

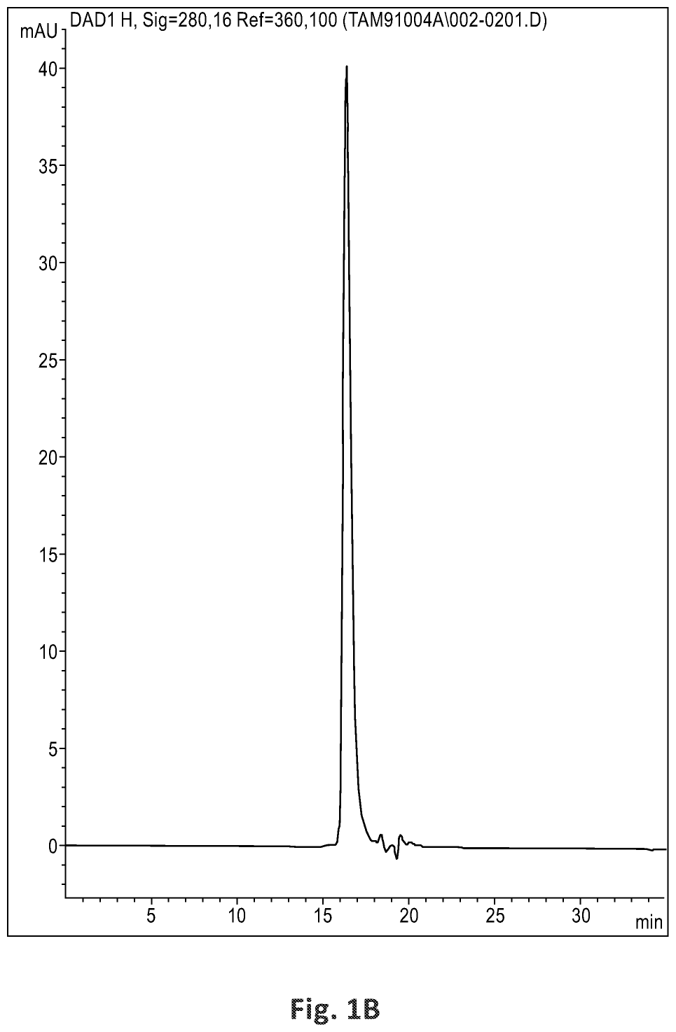

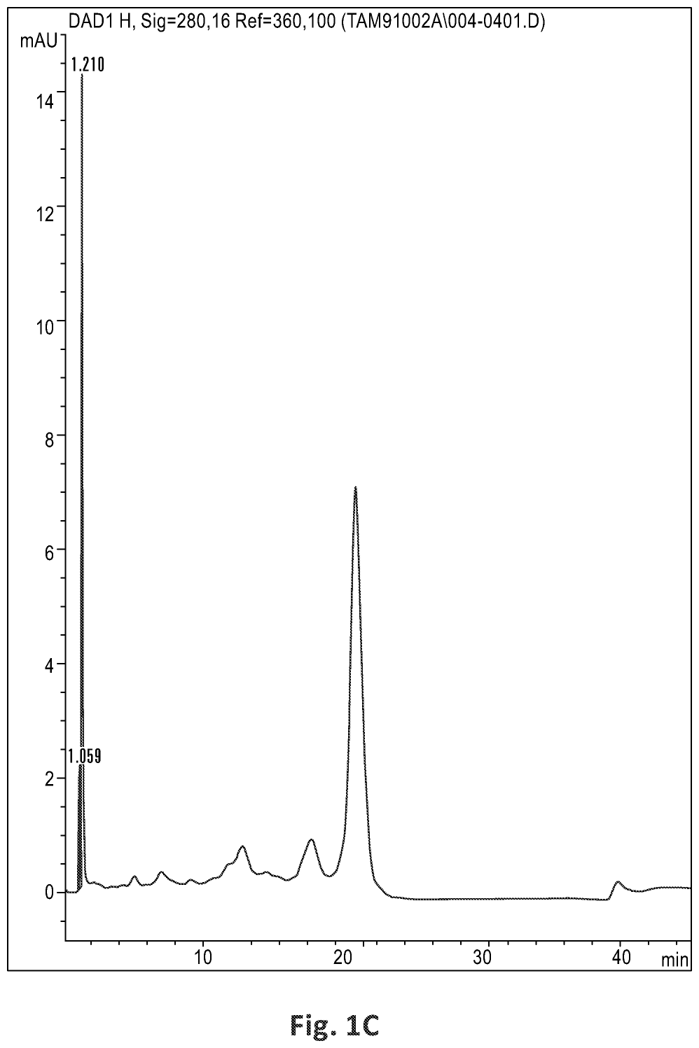

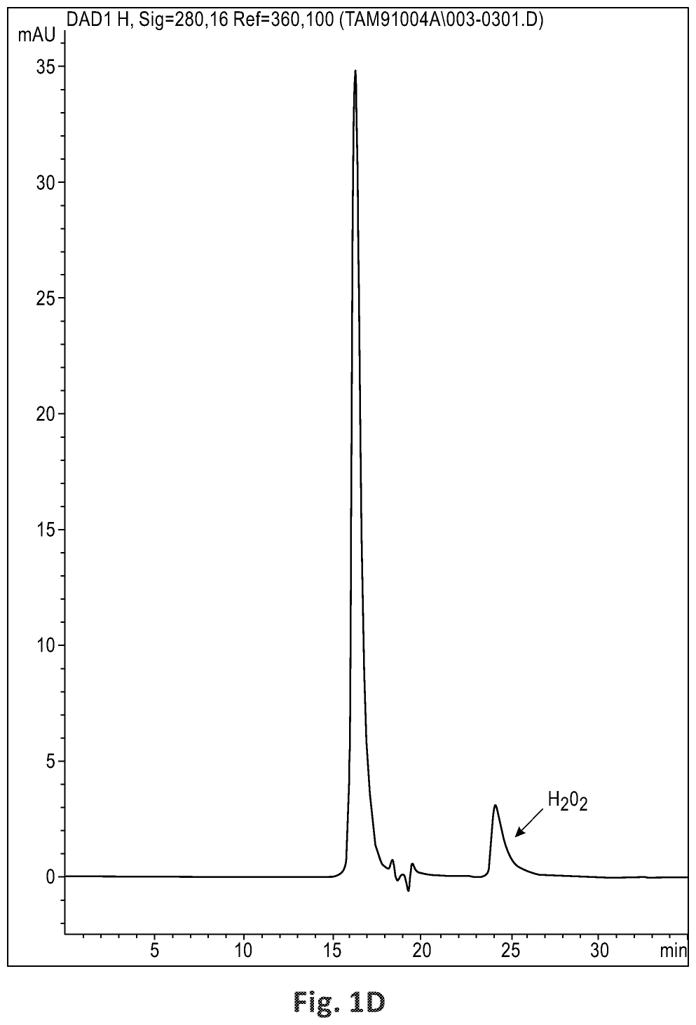

The linearity for the CEC was tested from 0.25 to 1 mg/mL (25 μL injection) and from to 0.1 mg/mL (100 μL injection) with a correlation coefficient >0.999. The % RSD for the CEC method on repeat injections was <2%. The linearity for the SEC was tested from 0.25 to 1 mg/mL (25 μL injection) with a correlation coefficient >0.999. The % RSD for the SEC method on repeat injections was also <2%. Based on these results, these methods were considered to be acceptable for preformulation and formulation work. Sample chromatograms are shown in FIGS. 1A-1D.

### Example 2

### Initial Excipient Compatibility Screen

Lyophilized ranpirnase was reconstituted with 1 mL of water and used as the control. The pH for the Control was 8.0. Additional vials were reconstituted with 1 mL of excipient/degradant solutions. The solutions were assayed by CEC initially and after storage at 25° C. with light protection. The results are summarized in Table 2.

TABLE 2
Initial Excipient Compatibility Results.

| | Assay | Assay | Assay | |
|---|---|---|---|---|
| | (% of | (% of | (% of | |
| | initial), | initial), | initial), | |
| Solution | 1 day | 1 week | 2 weeks | Comments |
| Control | 100 | 10 | 101 | No significant |
| | | | | impurities after 2 weeks |
| 15% | 101 | 100 | 99.8 | No significant |
| propylene | | | | impurities after 2 weeks |
| glycol | | | | |
| 0.1% H2O2 | 101 | 64.3 | 35.3 | Significant impurities |
| (degradant) | | | | eluting at 7-15 minutes |
| 2% | 101 | 90.0 | Not | Significant impurities |
| polysorbate- | | | tested | eluting at 10-15 minutes |
| polysorbate | | | | |
| Polysorbate 80 | | | | |
| 15% | 97.3 | 67.3 | Not | Significant impurities |
| Transcutol ® | | | tested | eluting at 10-15 minutes |
| 15% PEG 400 | 97.7 | 78.8 | Not | Significant impurities |
| | | | tested | eluting at 10-15 minutes |

Solutions with water (control) and propylene glycol showed no significant changes after 2 weeks of storage. After 1 week of storage, there was significant degradation of solutions with peroxide, polysorbate 80, Transcutol (diethylene glycol monoethyl ether), and PEG 400. The impurities seen in the excipient solutions were consistent with those observed for degradant (peroxide).

Propylene glycol is not a source for peroxides. Polysorbates, Transcutol, and PEGs are known to contain trace levels of residual peroxides. While the polysorbate 80 solution showed the least degradation of the tested excipients, excipients with potential to contain residual peroxides were not considered for further development.

After the 1-week timepoint, a sample of the peroxide solution was assayed using the SEC method. A freshly reconstituted (water) lyophilized product was used as the control. The SEC method confirmed there was degradation with peroxide (86.3% of the control solution), however, there were no significant new peaks. Also, SEC assay showed less loss for the main peak compared to the CEC assay (86.3% versus 64.3%) after 1 week of storage with peroxide. Based on the lower sensitivity to degradation and inability to detect new impurities, no additional work was performed with the SEC method.

Chromatograms for the 0.1% peroxide solution assay after 1 week are shown in FIGS. 1C and 1D for both the CEC and SEC methods. Peroxide elutes near the injection front for CEC. For SEC, peroxide elutes late in the chromatogram due to its low molecular weight.

### Example 3

### Second Excipient Compatibility Screen—Excipients with Low Potential for Peroxides

Additional solutions of excipients with low potential for peroxides were screened in a second study. Lyophilized ranpirnase was reconstituted with 1 mL of water and used as the control. The pH for the Control was 8.0. Additional vials were reconstituted with 1 mL of excipient solutions. The solutions were assayed by CEC initially and after storage at 25° C. with light protection. The results are summarized in Table 3.

TABLE 3
Second Excipient Compatibility Results.

| | Assay | Assay | Assay | |
|---|---|---|---|---|
| | (% of | (% of | (% of | |
| | initial), | initial), | initial), | |
| Solution | 1 day | 1 week | 2 weeks | Comments |
| Control | 101 | 99.5 | 102 | No significant |
| | | | | impurities after 2 weeks |
| 15% Glycerol | 99.7 | 99.5% | 99.5 | No significant |
| | | | | impurities after 2 weeks |
| 12% Hexylene | 99.6 | 99.6% | 100 | No significant |
| glycol | | | | impurities after 2 weeks |
| 10% Ethanol | 102 | 99.4 | 99.7 | No significant |
| (alcohol, | | | | impurities after 2 weeks |
| undenatured) | | | | |

This second excipient screen confirmed the results of the first screen, as excipients with low potential for residual peroxides had acceptable compatibility with ranpirnase. While ethanol would not be considered an acceptable excipient for its irritation potential, it was evaluated to confirm the hypothesis that excipients with residual peroxides should be excluded from the formulation.

### Example 4

### Antimicrobial Screen

An aqueous based formulation would require an antimicrobial preservative(s). Solutions with antimicrobials were prepared and used to reconstitute the lyophilized product to 1 mg/mL and stored at 25° C. for 2 weeks. The results are summarized in Table 4.

TABLE 4
Antimicrobial Screen Results.

| | Assay | Assay | |
|---|---|---|---|
| | (% of | (% of | |
| | initial), | initial), | |
| Preservative Solution | 1 week | 2 weeks | Comments |
| Control (water) | 98.6 | 99.1 | No significant |
| | | | impurities after 2 weeks |
| 0.11% Methylparaben/ | 99.8 | 99.6 | No significant |
| 0.022% Propylparaben (in | | | impurities after 2 weeks |
| 10% propylene glycol) | | | |
| 1.0% Benzyl alcohol | Not | Not | Interference with |
| | tested | tested | chromatography at |
| | | | initial |
| 0.1% EDTA | 101 | 99.6 | No significant |
| | | | impurities after 2 weeks |

During initial testing by CEC, the benzyl alcohol peak interfered with peak for ranpirnase. No additional testing with benzyl alcohol were performed. The parabens and EDTA showed good compatibility with ranpirnase. EDTA also acts as a chelating agent for metal ions.

### Example 5

### Antioxidant Screen

Due to the sensitivity to oxidation, several antioxidants approved for topical drug products were evaluated. Solutions with antioxidants were prepared and used to reconstitute the lyophilized product to 1 mg/mL and stored at 25° C. for 10 days. The results are summarized in Table 5.

TABLE 5
Antioxidant Screen Results.

| | | Assay | |
|---|---|---|---|
| | Initial | (% of | |
| | assay, | initial), | |
| Antioxidant | mg/mL | 10 days | Comments |
| Water (control) | 1.0 | 99.2 | No significant |
| | | | impurities after 2 weeks |
| 0.1% H2O2 | 1.0 | 62.2 | Significant impurities |
| (negative control) | | | eluting at 7-15 minutes |
| 0.02% butylated | 1.0 | 62.4 | Significant impurities |
| hydroxyanisole (BHA) | | | eluting at 7-15 minutes |
| (in 30% propylene | | | |
| glycol) | | | |
| 0.2% Ascorbic acid | Not | Not | No impurities observed |
| (pH 5.0) | detected | tested | |
| 0.1% Propyl gallate | 1.0 | 45.7 | Significant impurities |
| (in 30% propylene | | | eluting at 7-15 minutes |
| glycol) | | | |

The controls in this screening study behaved similarly to controls in an earlier study. Ascorbic acid may have ion paired with ranpirnase to alter the chromatography significantly. No attempts were made to investigate the details of the issue. Further, BHA and propyl gallate accelerated the degradation of ranpirnase. Based on these results, the use of an antioxidant does not appear to be a promising route to a suitable formulation.

### Example 6

### pH Adjustment

Having a pH significantly above 7 can difficult hydrolysis of many excipients. To evaluate the effect of a pH adjustment to 6.5-7.0, solutions of ranpirnase were prepared in this pH range using 10 mM acetate, citrate, and phosphate buffers. The results are summarized in Table 6.

TABLE 6
pH Adjustments Results.

| | Assay | Assay | |
|---|---|---|---|
| | (% of | (% of | |
| | initial), | initial), | |
| Buffer | 2 weeks | 4 weeks | Comments |
| Control (water) | 99.2 | 98.6 | No significant |
| | | | impurities after 4 weeks |
| Phosphate | 100 | 98.2 | No significant |
| | | | impurities after 4 weeks |
| Citrate | 101 | 102 | No significant |
| | | | impurities after 4 weeks |
| Acetate | 99.7 | 101 | No significant |
| | | | impurities after 4 weeks |

All three buffers were comparable to the control for assay value by CEC after 4 weeks at 25° ° C. Since the citrate had a pK closest to the target pH range, it was selected for formulation studies.

### Example 7

### Surfactant Physical Compatibility

Excluding excipients with potential residual peroxides would exclude almost all of the surfactants commonly used in cream and lotion formulations. Most surfactants have polar head groups based on polyethylene oxide or POE. Sucrose stearate (SS) is approved in topical drug products and does not have a POE head group. However, if there are buffer salts in the formulation, SS requires a more polar cosurfactant to form stable oil-in-water emulsions. Without a POE cosurfactant, ionic surfactants such as sodium dodecyl sulfate (SDS) or sodium lauroyl sarcosinate (SLS) can be used. Both SDS and SLS are approved topical drug products.

Physical compatibility of these surfactants was screened by reconstituting the lyophilized powder with various surfactant combinations and observed for several days. These results are summarized in Table 7.

TABLE 7
Surfactant Physical Compatibility Results.

| Surfactant | Appearance | Appearance | |
|---|---|---|---|
| Solution | at 20° C. | at 5° C. | Comments |
| 0.25% SDS | Clear | Some | ~48 hours for ppt to |
| | | precipitate | appear |
| 0.25% SLS | Some | Clear | Room temp. sample never |
| | precipitate | | completely cleared after |
| | | | reconstitution |
| 0.25% SDS | Clear | Clear | Promising combination |
| 1% SS | | | |
| 0.25% SLS | Very | Clear | Initially clear followed by |
| 1% SS | slight haze | | slight haze |

Based on the physical compatibility study, the SDS/SS combination was selected as the surfactant system for emulsion formulations.

### Example 8

### Ranpirnase Stability at 55° C.

During compounding, the aqueous and oil phases for creams would require heating to ˜55° C. for brief period (45-60 min). An aliquot of 4 mg/mL ranpirnase solution was stored at 50-55° C. and assayed at various time points. The results are summarized in Table 8.

TABLE 8
Ranpirnase stability at 55° C.

| Time at 50-55° C. | Assay, % of initial | Comments |
|---|---|---|
| 70 min | 97.8 | No significant impurities |
| 2 hours | 97.6 | No significant impurities |
| Overnight (~15 hours) | 97.1 | No significant impurities |

Solution stability of the 4 mg/mL solution appeared to be stable at high temperature for a time that would be sufficient for cream compounding.

### Example 9

### Ranpirnase Formulations

Examples 1-7 show that:
a. Ranpirnase is sensitive to peroxide and excipients that can contain and/or generate them.
b. Approved topical antioxidants decreased ranpirnase stability at 25° C.
c. Parabens and EDTA were compatible antimicrobial agents.
d. Ranpirnase was stable at pH 6.5-7.0 for up to 4 weeks at 25° C. Citrate was chosen as the buffer base.
e. SDS and SS would be used as surfactants for cream formulations.

Cream and gel bases were screened and the structure forming ingredient selection is summarized in Table 9.

TABLE 9
Cream and Gel Vehicles.

| Vehicle | | |
|---|---|---|
| base | Structure forming ingredient(s) | Rationale |
| Gel | Hydroxyethyl cellulose or HEC | Gelling agent for K-Y Jelly, |
| | (Natrosol 250 HXX) 1.75% w/w | potential to stabilize proteins. |
| Cream | SDS/SS with cetostearyl alcohol | POE-free emulsifier system. |

The composition of the investigated gels and creams are summarized in Table 10 and Table 11, respectively.

TABLE 10
Gels (All numbers in % w/w).

| | Gel 1 | Gel 2 | Gel 3 |
|---|---|---|---|
| Hydroxyethylcellulose | 1.75 | 1.75 | 1.75 |
| EDTA | 0.1 | 0.1 | 0.1 |
| Methylparaben | 0.11 | 0.11 | 0.11 |
| Propylparaben | 0.022 | 0.022 | 0.022 |
| Glycerol | 10.0 | 5.0 | 5.0 |
| Propylene glycol | 0.0 | 5.0 | 0.0 |
| Hexylene glycol | 0.0 | 0.0 | 5.0 |
| Purified water | q.s. 100% | q.s. 100% | q.s. 100% |

TABLE 11
Creams (All numbers in % w/w).

| | Cream 1 | Cream 2 | Cream 3 |
|---|---|---|---|
| Sucrose stearate | 1.5 | 1.5 | 1.5 |
| Sodium dodecyl sulfate | 0.2 | 0.2 | 0.2 |
| Cetostearyl alcohol | 8.0 | 8.0 | 8.0 |
| Light mineral oil | 5.0 | 0.0 | 0.0 |
| Octyldodecanol | 0.0 | 5.0 | 12.0 |
| EDTA | 0.1 | 0.1 | 0.1 |
| Methylparaben | 0.15 | 0.15 | 0.15 |
| Propylparaben | 0.05 | 0.05 | 0.05 |
| Glycerol | 7.5 | 7.5 | 7.5 |
| Purified water | q.s. 100% | q.s. 100% | q.s. 100% |

Since a modified buffer would be required for the active formulations, to adjust the buffering in the 4 mg/mL ranpirnase frozen solution, no buffer salts were added to the vehicle formulations. The EDTA salt can provide sufficient buffering capacity for pH 6.5-7.0 in these vehicles.

Vehicle Compounding. Vehicles were compounded at the 200 g scale. Compounding for the gels and creams are summarized below.

Gel 1 (Glycerol was not a suitable vehicle for adding parabens) was prepared according to the following protocol:
1. In the Main Vessel, add water and glycerol and start heating to 70° C. Mix with a propeller blade on an overhead mixer until homogeneous. Add parabens while heating.
2. Parabens will dissolve at 65-70° C., once that occurs start cooling to room temperature.
3. When contents reach room temperature, check vessel and replace any evaporated water.
4. Add EDTA and mix until dissolved.
5. Add HEC to Main Vessel with sufficient mixing to disperse the powder. Increase mixing speed to keep the gel moving while the HEC hydrates/thickens.
6. Replace propeller blade with anchor blade. Mix the formulation for at least 60 min. to allow hydration of the HEC. Use a stainless steel spatula for side scraping if needed.

Gels 2 and 3 (Blending propylene or hexylene glycol with glycerol facilitates paraben addition at room temperature) were prepared according to the following protocol:
1. In the Main Vessel, add water (reserve 5% for rinsing) and EDTA. Mix with a propeller blade on an overhead mixer until homogeneous.
2. Add glycerol and propylene glycol or hexylene glycol to the Parabens Vessel. Mix with a stir bar, add parabens, and mix until dissolved.
3. Add contents of Parabens Vessel to Main Vessel. Add reserved rinse water to Parabens Vessel, mix, then add to Main Vessel.
4. Add HEC to Main Vessel with sufficient mixing to disperse the powder. Increase mixing speed to keep the gel moving while the HEC hydrates/thickens.
5. Replace propeller blade with anchor blade. Mix the formulation for at least 60 min. to allow hydration of the HEC. Use a stainless-steel spatula for side scraping if needed.

Creams were prepared according to the following protocol:
1. In the Main Vessel, add water, EDTA, glycerol and SDS. Mix with a propeller blade on an overhead mixer until homogeneous. Heat to 60-65° C. and maintain that temperature
2. In a separate Lipids Vessel, combine the oils (mineral oil or octyldodecanol), cetostearyl alcohol, and parabens. Heat to 60-65° C. and mix until the Lipids Vessel contents are uniform. Maintain temperature at 60-65° C.
3. Add Lipids Vessel's contents to the Main Vessel with propeller blade mixing. Mix the formulation for at least 10 min. Start high shear mixing using a 10 mm rotor/stator homogenizer.
4. Cool the Main Vessel while continuing high shear mixing. When the Main Vessel contents reach 45° C., stop high shear mixing and return to propeller mixing.
5. Continue mixing, using a stainless steel spatula for side scraping as needed, until it reaches 30° C.

### Example 10

### Vehicle Formulation Stability

Samples of the vehicles described in Example 9 were stored at 25° C. for 1 month. The initial and 1-month results are summarized in Table 12 and Table 13, respectively

TABLE 12
Initial Stability of Vehicles.

| | | Viscosity, | |
|---|---|---|---|
| Formulation | pH | cP | Appearance |
| Gel 1 | 6.69 | 31,500 | Clear to translucent soft gel |
| Gel 2 | 6.68 | 33,900 | Clear to translucent soft gel |
| Gel 3 | 6.83 | 32,000 | Clear to translucent soft gel |
| Cream 1 | 6.81 | 17,900 | White to off-white homogenous cream |
| Cream 2 | 6.60 | 18,900 | White to off-white homogenous cream |
| Cream 3 | 6.71 | 46,100 | White to off-white homogenous cream |

TABLE 13
Stability of Vehicles after 1-month.

| | | Viscosity, | |
|---|---|---|---|
| Formulation | pH | cP | Appearance |
| Gel 1 | 6.75 | 32,000 | Clear to translucent soft gel |
| Gel 2 | 6.89 | 33,100 | Clear to translucent soft gel |
| Gel 3 | 6.82 | 32,400 | Clear to translucent soft gel |
| Cream 1 | 6.67 | 18,300 | White to off-white homogenous cream |
| Cream 2 | 6.66 | 18,500 | White to off-white homogenous cream |
| Cream 3 | 6.76 | 47,500 | White to off-white homogenous cream |

There were no significant changes in pH, viscosity, or appearance for the formulations stored at 25° C. for 1 month. Samples of all nine formulations were also subjected to 3 freeze/thaw cycles: 3 days at −20° C. followed by 4 days at room temperature. They were evaluated for appearance after each cycle. No changes in appearance were observed. Based on these results, all six vehicles would be suitable for compounding with ranpirnase.

### Example 11

### Extraction Methods

Gel extraction was tested using the following steps for Gel 2:
1. Add ˜1.0 g of gel vehicle to a 10 mL volumetric flask.
2. Add 0.25 mL of 4 mg/mL ranpirnase frozen stock solution.
3. Add ˜5 mL of water to the vial and vortex to disperse/dissolve the gel.
4. Add a small stir bar and mix for 15-20 minutes.
5. Remove stir bar, bring flask to volume with water and mix.
6. Add an aliquot to an amber HPLC vial for analysis.

Cream extraction was tested using the following steps for Cream 3:
1. Add ˜1.0 g of cream vehicle to a 10 mL volumetric flask.
2. Add 0.25 mL of 4 mg/mL ranpirnase frozen stock solution.
3. Add ˜5 mL of water to the vial and vortex to disperse the cream.
4. Add a small stir bar and mix for 45-50 minutes. Check periodically to ensure there are no agglomerates of cream excipients. Any agglomerates should be dispersed by vortexing.
5. Remove stir bar, bring flask to volume with water and mix.
6. Bring flask to volume with HPLC diluent, mix, and filter an aliquot through a 0.45 micron nylon syringe filter into an amber HPLC vial for analysis.

Extraction experiments were performed in duplicate and the results are shown in in Table 14.

TABLE 14
Extraction Experiments Results.

| Vehicle | % recovery, Extraction 1 | % recovery, Extraction 2 |
|---|---|---|
| Gel 2 | 99.5 | 98.9 |
| Cream 3 | 98.5 | 98.8 |

All of the recovery results were between 98.5 and 99.5%. Based on these results, the extraction methods were considered to be suitable for analysis of prototype ranpirnase active formulations.

### Example 12

### Active Formulation Compounding

For active formulations, sodium citrate and HCl were used to adjust the pH of the solution of the 4 mg/mL frozen solution (thawed) to a target of 6.5. The compositions for the active gels and cream vehicles are summarized in Tables 15 and 16.

TABLE 15
Gel Formulations - 0.1% Ranpirnase Gels. All numbers in % w/w.

| | Gel 1 | Gel 2 | Gel 3 |
|---|---|---|---|
| 4 mg/L frozen | 25.0 | 25.0 | 25.0 |
| solution | | | |
| Hydroxyethylcellulose | 1.75 | 1.75 | 1.75 |
| EDTA | 0.1 | 0.1 | 0.1 |
| Sodium citrate | 0.42 | 0.42 | 0.42 |
| 10% HCl | 0.21 | 0.21 | 0.21 |
| Methylparaben | 0.11 | 0.11 | 0.11 |
| Propylparaben | 0.022 | 0.022 | 0.022 |
| Glycerol | 10.0 | 5.0 | 5.0 |
| Propylene glycol | 0.0 | 5.0 | 0.0 |
| Hexylene glycol | 0.0 | 0.0 | 5.0 |
| Purified water | q.s. 100% | q.s. 100% | q.s. 100% |

TABLE 16
Cream Formulations - 0.1% Ranpirnase
Gels. All numbers in % w/w.

| | Cream 1 | Cream 2 | Cream 3 |
|---|---|---|---|
| 4 mg/L frozen | 25.0 | 25.0 | 25.0 |
| solution | | | |
| Sucrose stearate | 1.5 | 1.5 | 1.5 |
| Sodium dodecyl | 0.2 | 0.2 | 0.2 |
| sulfate | | | |
| Cetostearyl alcohol | 8.0 | 8.0 | 8.0 |
| Light mineral oil | 5.0 | 0.0 | 0.0 |
| Octyldodecanol | 0.0 | 5.0 | 12.0 |
| Sodium citrate | 0.42 | 0.42 | 0.42 |
| 10% HCl | 0.21 | 0.21 | 0.21 |
| EDTA | 0.1 | 0.1 | 0.1 |
| Methylparaben | 0.15 | 0.15 | 0.15 |
| Propylparaben | 0.05 | 0.05 | 0.05 |
| Glycerol | 7.5 | 7.5 | 7.5 |
| Purified water | q.s. 100% | q.s. 100% | q.s. 100% |

There was a limited amount of 4 mg/mL frozen solution available. 4 vials (10-12 mL each) were sent prior to initiating preformulation work. Four additional vials were sent afterwards. One vial was used in preformulation studies.

The batch size would need to be scaled down to ˜40-45 g. At this small scale, 1 vial could be used per batch. That gave enough material to make 6 active batches with one vial in reserve. The gels could be manufactured at this scale with no significant change to the process outline above for the gel vehicles.

However, creams could not be compounded at the 40-45 g scale using the vehicle cream process described above. Using a smaller 7 mm rotor-stator homogenizer would require a batch size >50-55 g to prevent excessive splashing and product aeration. So, the emulsion compounding was scaled down using two 50 mL syringes (Exel International) and a 3-way stopcock (Medex), according to the following protocol:
1. The sucrose stearate, cetostearyl alcohol, parabens, and oil (mineral oil or octyldodecanol) were combined, heated to ˜55-60° C., and mixed until homogeneous.
2. The oil phase from Step 1 as drawn into a 50 mL pre-heated syringe and stored in a 55-60° C. oven until used.
3. The water-soluble excipients and the API stock solution were combined in a separate vessel. The contents were heated to 55-60° C.
4. The aqueous phase from Step 3 was drawn into a pre heated 50 mL syringe and connected to one port of a pre-heated stopcock.
5. The oil phase syringe was removed from the oven and connected to a second port of the stopcock. The stopcock's valve was adjusted so that the third port was blocked off and the oil and aqueous syringes were connected.
6. The syringe plunger for the aqueous phase was firmly depressed and rapidly expelled the contents in the oil phase. The oil phase plunger was immediately depressed expelling all of the formulation contents into the other syringe.
7. This process was repeated until the formulation cooled to ˜40° C. and had thickened.
8. The contents were added to a beaker with a small stir bar to continue mixing the emulsion until it fully thickened.

Vehicle versions of Cream 1 and Cream 3 were successfully prepared using this technique. Initial results on active creams are discussed in the next section.

From the current experiments it was inferred that ranpirnase might need to be concentrated to a higher level, as the the dilute 4 mg/mL frozen solution is not suitable for large scale compounding. Concentration techniques for the frozen solution could include diafiltration and/or lyophilization.

### Example 13

### Initial Results for Active Creams with SDS

Cream 1 and 3 were successfully prepared at the 40-45 g scale using the syringe/stopcock combination to generate high shear on a small scale. These creams were extracted and assayed for ranpirnase to confirm that this was a suitable process/formulation base prior to making an additional cream. Ranpirnase was not detectable in either cream extract.

The extraction time was doubled and had no effect on improving recovery. These results could have been due to degradation of the active during compounding. However, the ranpirnase solution showed acceptable stability at elevated compounding temperatures for a duration much longer than the compounding time. Since there is an ionic interaction between ranpirnase and SDS, the API could have become resistant to extraction based on the presence of an oil phase and a hydrophobic waxy cosurfactant (cetostearyl alcohol). Using a stronger organic-based solvent may have improved recovery but, would have made analysis by CEC challenging.

To determine if SDS was the issue, the creams were reformulated to remove SDS and SLS and replace this surfactant system with polysorbate 60. To minimize the potential for residual peroxides in this POE surfactant, Croda's Super Refined grade was used. SDS and SLS would be removed and replaced with 1.5% polysorbate 60 SR (super refined). Water would be used to q.s. the formulation to 100%.

The modified Cream 3 was successfully prepared using the small scale

emulsification technique. The cream was slightly thin, so 1% HEC was added and manually mixed into the formulation. This successfully thickened the cream which was designated as Cream 3a. The analysis of a Cream 3a extract showed significantly improved recovery. The switch to polysorbate 60 SR appeared to be a promising direction.

Since Cream 3 was the thickest of the cream formulations, making Creams 1 and 2 with polysorbate 60 was considered too risking with a limited supply of ranpirnase solution. The Cream 3 base was modified to make two potentially thicker creams (3b and 4) and their compositions are summarized in Table 17.

TABLE 17
Compositions of Creams 3a, 3b, and 4.

| | Cream 3a | Cream 3b | Cream 4 |
|---|---|---|---|
| 4 mg/L frozen | 25.0 | 25.0 | 25.0 |
| solution | | | |
| Polysorbate 60 SR | 1.5 | 1.5 | 1.5 |
| Cetostearyl alcohol | 8.0 | 12.0 | 12.0 |
| Light mineral oil | 0.0 | 0.0 | 5.0 |
| Octyldodecanol | 12.0 | 5.0 | 12.0 |
| Sodium citrate | 0.42 | 0.42 | 0.42 |
| 10% HCl | 0.21 | 0.21 | 0.21 |
| EDTA | 0.1 | 0.1 | 0.1 |
| Methylparaben | 0.15 | 0.15 | 0.15 |
| Propylparaben | 0.05 | 0.05 | 0.05 |
| Glycerol | 7.5 | 7.5 | 7.5 |
| Hydroxyethylcellulose | 1.0 | 0.0 | 0.0 |
| Purified water | q.s. 100% | q.s. 100% | q.s. 100% |

Creams 3b and 4 were successfully prepared. Initial results, plus those for the gels are summarized in Example 12. Portions of each gel and cream active batch were filled into Type 1 glass vials with a butyl rubber stopper and aluminum crimp closure. Vials were stored at 2-8 and 25° C. The batch sizes were too small to allow for viscosity measurements.

### Example 14

### Initial Results for Gels and Creams

The initial results for active gels and creams are summarized in Table 18.

TABLE 18
Initial Results.

| Formulation | Assay, % w/w | pH | Appearance | Impurities |
|---|---|---|---|---|
| Gel 1 | 0.069 | 6.58 | Conforms¹ | Not Detected |
| Gel 2 | 0.089 | 6.61 | Conforms¹ | Not Detected |
| Gel 3 | 0.094 | 6.62 | Conforms¹ | Not Detected |
| Cream 3a | 0.095 | 6.55 | Conforms² | Not Detected |
| Cream 3b | 0.083 | 6.60 | Conforms² | Not Detected |
| Cream 4 | 0.087 | 6.55 | Conforms² | Not Detected |

¹Clear to translucent soft gel.
²White to off-white homogenous cream

The initial assay results were lower than the nominal target of 1 mg/mL or 0.1% w/w. The frozen solution assay was accepted at its nominal value of 4 mg/mL. No assay was performed since any adjustments would likely require higher levels of frozen solution (i.e., smaller batch sizes) and the batch sizes were already very small for prototype active semisolid formulations.

### Example 15

### Stability Data for Gels and Creams

The stability results for gels and creams are summarized in Table 19 and Table 20.

TABLE 19
Stability of Gels Results.

| | Gel 1 | Gel 2 | Gel 3 |
|---|---|---|---|
| 1 month, 25° C. | | | |
| Assay, % w/w | 0.074 | 0.085 | 0.083 |
| Impurities, area % | 8.8 | 4.6 | 3.9 |
| Appearance | Conforms¹ | Conforms¹ | Conforms¹ |
| 3 month, 25° C. | | | |
| Assay, % w/w | 0.054 | 0.073 | 0.074 |
| Impurities, area % | 28.2 | 13.6 | 15.3 |
| Appearance | Conforms¹ | Conforms¹ | Conforms¹ |
| 3 month, 5° C. | | | |
| Assay, % w/w | 0.075 | 0.094 | 0.093 |
| Impurities, area % | Not Detected | Not Detected | Not Detected |
| Appearance | Conforms¹ | Conforms¹ | Conforms¹ |

¹Clear to translucent soft gel.

TABLE 20
Stability of Creams Results.

| | Cream 3a | Cream 3b | Cream 4 |
|---|---|---|---|
| 1 month, 25° C. | | | |
| Assay, % w/w | 0.062 | 0.075 | 0.070 |
| Impurities, area % | 12.9 | 13.7 | 15.5 |
| Appearance | Conforms¹ | Conforms¹ | Conforms¹ |
| 1 month, 5° C. | | | |
| Assay, % w/w | 0.100 | 0.096 | 0.091 |
| Impurities, area % | Not Detected | Not Detected | Not Detected |
| Appearance | Conforms¹ | Conforms¹ | Conforms¹ |
| 3 months, 25° C. | | | |
| Assay, % w/w | 0.041 | 0.055 | 0.049 |
| Impurities, area % | 35.1 | 32.0 | 33.8 |
| Appearance | Conforms¹ | Conforms¹ | Conforms¹ |
| 3 months, 5° C. | | | |
| Assay, % w/w | 0.092 | 0.095 | 0.093 |
| Impurities, area % | Not Detected | Not Detected | Not Detected |
| Appearance | Conforms¹ | Conforms¹ | Conforms¹ |

¹White to off-white homogenous cream

The gels showed no significant degradation after 3 months at 5° C., no significant trend down in assay values and no new impurities observed. However, after 1 and 3 months at 25° C., the assay values showed a significant trend downward that was accompanied by an upward trend in new impurities detected. Interestingly, Gels 2 and 3 had lower impurities levels than Gel 1 after 1 month and this trend continued to the 3 month timepoint. Gel 2 (propylene glycol) and Gel 3 (hexylene glycol) both contained semipolar solvents in addition to glycerol. It's possible these solvents were able to interact with hydrophobic portions of ranpirnase and stabilize the molecule more effectively at room elevated temperature.

The creams showed no significant degradation after 1 or 3 months at 5° C., no significant trend in assay values and no new impurities observed. However, after 1 and 3 months at 25° C., the assay values showed a significant trend downward that was accompanied by an upward trend in new impurities detected. There did not appear to be a significant difference in ranpirnase stability between the three cream formulations. The difference between the three creams was the composition of the oil phase. Since ranpirnase is very water soluble, this might explain why the stability results were consistent across the three cream formulations.

### Example 16

### Method Development for Enzymatic Activity Assay for Ranpirnase

In order to assess the method accuracy, three spike studies were analyzed against the calibration curve. The recovery was found to be ±20% from the expected value. The R² of the standard curve was above 0.98. The recovery results can be seen in the Table 21.

TABLE 21
Recovery in different samples.

| | Concentration | | % |
|---|---|---|---|
| Sample | (0.6 μM) | Absorbance | recovery |
| Cream Positive 0.6 μM | 0.57 | 0.360 | 0.95 |
| Gel 1 Positive 0.6 μM | 0.65 | 0.425 | 1.08 |
| Gel 2 Positive 0.6 μM | 0.63 | 0.407 | 1.05 |
| Gel 3 Positive 0.6 μM | 0.58 | 0.367 | 0.97 |

The ranpirnase standards were prepared according to the table 22.

TABLE 22
Preparation of ranpirnase standards.

| | | | | HSA 0.2 |
|---|---|---|---|---|
| | | | | mg/ml |
| | | | Ranpirnase | Working |
| | Working | | solution | solution |
| | solution | Sample stock | (μl) | (μl) |
| Stock | 8.64 μM Stock | 84.6 μM stock | 100 | 900 |
| Working | 8.64 μM Stock | 84.6 μM stock | 100 | 900 |
| standard | | | | |
| | 8.64 μM Stock | 84.6 μM stock | 100 | 900 |
| | 8.64 μM Stock | 84.6 μM stock | 100 | 900 |
| | 8.64 μM Stock | 84.6 μM stock | 100 | 900 |
| | 8.64 μM Stock | 84.6 μM stock | 100 | 900 |
| | 8.64 μM Stock | 84.6 μM stock | 100 | 900 |

### Example 17

### In Vitro Dermal Permeation and Penetration of Ranpirnase Topical Formulations

The In Vitro permeation and penetration study in Franz diffusion cells was using dermatomed human cadaver skin, human epidermis, and a mesoporous membrane of polyvinylidene difluoride. The study was testing two strength concentrations of Ranpirnase, 1.3 mg/ml (“low”) and 2.1 mg/ml (“High”).

The goal of this study was to test the release of ranpirnase from the gel formulation and its penetration into the skin layers.

Arm 1—Permeation and deposition of Ranpirnase from low and high formulations using human cadaver skin in 6-fold replications are performed according to the below protocol-
Receptor well sampling at 8, 24 and 48 hours;
Optionally, receptor well sampling at 4 hours;
At 48 hours the skin is gently washed with 200 μl water:ethanol 50:50 volume ratio, the solution is allowed to contact skin surface for 5 minutes, the solution is removed and pat dried with KimWipe, one low and on high wash samples of the 12 replicated is saved.
One of the washings from each formulation is saved as a positive control;
After washing, the skin is tapestripped ten (10) times;
The tapestrips are collected and bined into four (4) groups: TS1, TS2, TS3-6 and TS7-10;
The epidermis is separated from the dermis and extracted from each.

Arm 2—Penetration of Ranpirnase from low and high formulations through human cadaver epidermis in 4 replicates is performed according to the below protocol-
Just prior to the study, human cadaver skin is heat split to separate the stratum corneum and epidermis from the dermis;
The stratum corneum and epidermis is than used in Franz Diffusion cells;
Receptor well is sampled at 8, 24 and 48 hours.

Arm 4—Penetration of Ranpirnase from low and high formulations through the porous membrane PVDF in 3-fold replications is performed according to the below protocol-
Receptor well is sampled at 8, 24 and 48 hours;

The concentration of ranpirnase in the test samples was measured using an immunoassay and is presented in table 23.

TABLE 23
penetration of ranpirnase through different skin layers.

| | Gel | | Concen- |
|---|---|---|---|
| | concen- | | tration |
| Arm | tration | Sample | (pg/mL) |
| Arm 1 | High | TS1 | 133,000 |
| Arm 1 | High | TS2 | 5,300 |
| Arm 1 | High | TS3 | 2,600 |
| Arm 1 | High | TS4 | 1,400 |
| Arm 1 | High | 8 hr receptor fluid (full thickness) | BQL |
| Arm 1 | Low | 8 hr receptor fluid (full thickness) | BQL |
| Arm 1 | High | 24 hr receptor fluid (full thickness) | 1,100 |
| Arm 1 | Low | 24 hr receptor fluid (full thickness) | 82 |
| Arm 2 | High | 8 hr receptor fluid (Stratum | 5,300 |
| | | corneum + epidermis) | |
| Arm 2 | Low | 8 hr receptor fluid (Stratum | 2,100 |
| | | corneum + epidermis) | |
| Arm 2 | High | 24 hr receptor fluid (Stratum | 58.000 |
| | | corneum + epidermis) | |
| Arm 2 | Low | 24 hr receptor fluid (Stratum | 52 |
| | | corneum + epidermis) | |
| Arm 4 | High | 24 hr receptor fluid (PVDF | 1,800,000 |
| | | membrane) | |
| Arm 4 | Low | 24 hr receptor fluid (PVDF | 693,000 |
| | | membrane) | |

BQL—Below limit of quantitation < 11.0 pg/mL

Ranpirnase was released from the formulation as shown by the penetration across the PVDF membrane. Ranpirnase penetrates the stratum corneum and epidermis skin layers and to some extent also full thickness skin.

### Example 18

### Stability Analysis of Ranpirnase Gel Formulations

The ranpirnase gel was manufactured for preclinical studies (28-days dermal toxicology) at two strengths (0.1% and 0.31%), according to the composition described in Table 24. Both batched were produced on 600g scale. The gels were tested on release and following the 28-days study (˜3 months after manufacturing stored at 5 C). Both gels met specifications, although some degradation was observed in the high strength (0.31%) gel.

TABLE 24
Ranpirnase Topical Gel Composition for 0.1% w/w Strength.

| | | Composition |
|---|---|---|
| Ingredient | Purpose | (% w/w) |
| Ranpirnase BDSS ^{b} | Active | 25.0 |
| Glycerin, 99.7% USP | Solvent | 5.0 |
| Propylene Glycol, USP | Solvent | 5.0 |
| Hydroxyethyl Cellulose, NF | Thickening agent | 1.75 |
| Sodium Citrate, Dihydrate, USP | pH adjustment | 0.42 ^{c} |
| 10% Hydrochloric Acid, NF | pH adjustment | 0.21 ^{c} |
| Methylparaben, NF | Preservative | 0.11 |
| Edetic Acid, NF | Preservative, | 0.10 |
| | chelating agent | |
| Propylparaben, NF | Preservative | 0.022 |
| Purified Water, USP | Solvent | Q.S. to 100% |

^{b} Bulk drug substance solution (BDSS) consists of 4.0 ± 2.0 mg/mL ranpirnase in 75 mM ammonium bicarbonate buffer.
^{c} Amount may vary; used to adjust the thawed BDSS to a target pH of 6.5.

For the 0.31% gel, BDSS at concentration of 3.576 mg/mL was used. The amount of BDSS was 88.3% all other ingredients identical to the described in Table 24.

The stability results are described in tables 25-28 below.

TABLE 25
0.1% gel, time 0 (release).

| Test | Method | Specification | Result |
|---|---|---|---|
| Appearance | Visual | Clear to trans- | Meets |
| | | lucent gel, free of | specification |
| | | foreign particles | |
| Viscosity | SOP QI-1166 | Report results | Top: 6.871 cPs |
| | DV3-HB | | Bottom: 7.613 cPs |
| | CP40 spindle | | Average: 7.242 cPs |
| | Speed: 3.0 RPM | | |
| | Time: 3 min | | |
| | Size: 0.6 g | | |
| pH (neat) | SOP-QI-020 | Report results | Top: 7.8 |
| | | | Bottom: 7.9 |
| | | | Average: 7.9 |
| Strength | Cation Exchange | 80-120% | Top 102.3% |
| (protein | HPLC | | Bottom 99.9% |
| contents) | | | |
| Purity | Cation Exchange | Not less than 95% | 100% |
| | HPLC | | |
| Related | Cation Exchange | Not more than | Non detected |
| substances | HPLC | 5% | |

TABLE 26
0.1% gel, about 3 months.

| Test | Method | Specification | Result |
|---|---|---|---|
| Appearance | Visual | Clear to trans- | Meets |
| | | lucent gel, free of | specification |
| | | foreign particles | |
| Viscosity | SOP QI-1166 | Report results | Top: 6.366 cPs |
| | DV3-HB | | Bottom: 5.921 cPs |
| | CP40 spindle | | Average: 6.144 cPs |
| | Speed: 3.0 RPM | | |
| | Time: 3 min | | |
| | Size: 0.6 g | | |
| pH (neat) | SOP-QI-020 | Report results | Top: 7.8 |
| | | | Bottom: 8 |
| | | | Average: 7.9 |
| Strength | Cation Exchange | 80-120% | Top 90.4% |
| (protein | HPLC | | Bottom 89.3% |
| contents) | | | |
| Purity | Cation Exchange | Not less than 95% | 100% |
| | HPLC | | |
| Related | Cation Exchange | Not more than | Non detected |
| substances | HPLC | 5% | |

TABLE 27
0.31% gel, time 0 (release).

| Test | Method | Specification | Result |
|---|---|---|---|
| Appearance | Visual | Clear to trans- | Meets |
| | | lucent gel, free of | specification |
| | | foreign particles | |
| Viscosity | SOP QI-1166 | Report results | Top: 7.116 cPs |
| | DV3-HB | | Bottom: 7.133 cPs |
| | CP40 spindle | | |
| | Speed: 3.0 RPM | | Average: 7.125 cPs |
| | Time: 3 min | | |
| | Size: 0.6 g | | |
| pH (neat) | SOP-QI-020 | Report results | Top: 8.0 |
| | | | Bottom: 8.1 |
| | | | Average: 8.1 |
| Strength | Cation Exchange | 80-120% | Top 114.8% |
| (protein | HPLC | | Bottom 115.0% |
| contents) | | | |
| Purity | Cation Exchange | Not less than 95% | 100% |
| | HPLC | | |
| Related | Cation | Exchange | Not more than 5% |
| substances | HPLC | | |

TABLE 28
0.31% gel, about 3 months.

| Test | Method | Specification | Result |
|---|---|---|---|
| Appearance | Visual | Clear to trans- | Meets |
| | | lucent gel, free of | specification |
| | | foreign particles | |
| Viscosity | SOP QI-1166 | Report results | Top: 6.087 cPs |
| | DV3-HB | | Bottom: 6.810 cPs |
| | CP40 spindle | | Average: 6.449 cPs |
| | Speed: 3.0 RPM | | |
| | Time: 3 min | | |
| | Size: 0.6 g | | |
| pH (neat) | SOP-QI-020 | Report results | Top: 8.3 |
| | | | Bottom: 8.2 |
| | | | Average: 8.3 |
| Strength | Cation Exchange | 80-120% | Top 105.7% |
| (protein | HPLC | | Bottom 105.6% |
| contents) | | | |
| Purity | Cation Exchange | Not less than 95% | 96% |
| | HPLC | | |
| Related | Cation Exchange | Not more than | 3.9% |
| substances | HPLC | 5% | |

### Example 19

### Toxicology Analysis of Rampirnase Gel Formulations

The 0.1% and 0.31 ranpirnase gels were tested in a non GLP 28 days toxicology study in minipigs. The test materials were administered once daily for 28 days during the study via topical application. The dose concentrations were 0.1 and 0.31% w/w and were administered at a dose volume of 0.75 mL/kg; this had been previously shown to represent the maximum feasible dose volume.

Ranpirnase gel, when applied topically to the anogenital region once daily for 28 days to Göttingen Minipig® at 0.1 or 0.31% w/w, was well tolerated with no definitive test article related findings. Based on these, the maximum tolerated dose (MTD) was at least 0.31% w/w, the highest dose evaluated, applied using the maximum feasible dose volume of 0.75 mL/kg.

### SUMMARY AND CONCLUSIONS

Examples 1-19 indicate the following:
1. Ranpirnase is sensitive to peroxide and excipients that could contain/generate them.
2. Approved topical antioxidants decreased ranpirnase stability at 25° C.
3. The CEC method showed new impurities in forced degradation studies. The SEC method showed a slight decrease in assay value, but no know impurities.
4. Parabens and EDTA were compatible antimicrobial agents that did not interfere with the CEC method.
5. Ranpirnase was stable at pH 6.5-7.0 for up to 4 weeks at 25° C. Citrate was chosen as the buffer base.
6. The 4 mg/mL ranpirnase frozen solution, after thawing, was stable for up to 15 hours at 55° C. This indicated that cream formulations should not show significant degradation during compounding at significantly shorter time durations at elevated temperatures.
7. All of the gel formulations were stable for up to 3 months at 5° C. Gels with propylene glycol or hexylene glycol were more stable at 25° C. than a gel with glycerol alone.
8. All of the cream formulations were stable for up to 3 months at 5° C. However, their stability at 25° C. was lower than that for the gel. All the cream formulations used glycerol as a hydrophilic solvent. Any future work on the cream base should include evaluation of semipolar solvents like propylene glycol and hexylene glycol.
9. Ranpirnase is released from a gel formulation and is capable of penetrating the stratum corneum and epidermis skin layers.
10. The maximum tolerated dose (MTD) of ranpirnase in a gel formulation is at least 0.31%, applied using the maximum feasible dose volume of 0.75 ml/kg.

1. A pharmaceutical composition comprising ranpirnase and a polyol.

2. The composition of claim 1, wherein said polyol is selected from a group comprising hexylene glycol, propylene glycol, glycerol, glycerol monolaureate, and ethylene glycol, or any combination thereof.

3. The composition of claim 1, wherein said polyol comprises about 10% of the weight of said pharmaceutical composition.

4. The composition of claim 2, wherein said glycerol comprises about 5%, and said propylene glycol comprises about 5% of the weight of said pharmaceutical composition.

5. The composition of claim 2, wherein said glycerol comprises about 5%, and said hexylene glycol comprises about 5% of the weight of said pharmaceutical composition.

6. The composition of claim 1, wherein said composition is stable at room temperature.

7. The composition of claim 6, wherein said composition has increased stability compared to a composition comprising ranpirnase and about 10% glycerol of the weight of said pharmaceutical composition.

8. The composition of claim 1, wherein said composition is formulated as a gel or as a cream.

9. The composition of claim 1, wherein said ranpirnase is at a concentration of about 0.1% w/w.

10. The composition of claim 1, wherein said composition is formulated for topical or dermal application.

11. The pharmaceutical composition of claim 1, further comprising an antimicrobial agent.

12. The pharmaceutical composition of claim 11, wherein said antimicrobial agent is selected from the group comprising methylparaben, propylparaben, EDTA, or any combination thereof.

13. The pharmaceutical composition of claim 1, wherein said composition does not comprise an antioxidant agent.

14. The pharmaceutical composition of claim 1, wherein said composition comprises a pH in the range of 6.5 to 7.

15. The pharmaceutical composition of claim 1, further comprising a surfactant agent.

16. (canceled)

17. The pharmaceutical composition of claim 1, further comprising hydroxyethyl cellulose (HEC).

18. (canceled)

19. A gel pharmaceutical composition comprising ranpirnase, glycerin, propylene glycol, hydroxyethyl cellulose, sodium citrate dihydrate, 10% hydrochloric acid, methylparaben, edetic acid, propylparaben and purified water, or comprising ranpirnase, hydroxyethylcellulose, EDTA, methylparaben, propylparaben, glycerol, and:
(a) propylene glycol, and/or
(b) hexylene glycol.

20. (canceled)

21. A cream pharmaceutical composition comprising ranpirnase, sucrose stearate, sodium dodecyl sulfate, cetostearyl alcohol, EDTA, and:
(a) light mineral oil, and/or
(b) octyldodecanol.

22. A method for treating a viral disease in a subject in need thereof, the method comprising administering the pharmaceutical composition of claim 1.

23. The method of claim 22, wherein said viral disease is selected from the group comprising a viral skin disease; herpes zoster; chickenpox; molluscum contagiosum; warts; measles; hand, foot and mouth disease, human papillomavirus (HPV) infection, or any combination thereof.
